(19) United States
(12) Patent Application Publication
(10) Pub. No.: US 2021/0220818 A1
(43) Pub. Date: Jul. 22, 2021

(54) METHOD FOR THE CAPTURE AND ISOLATION OF DISEASE MATERIAL FROM FLOWING MATTER

(71) Applicant: PATH EX, Inc., Houston, TX (US)

(72) Inventor: Sinead E. MILLER, Houston, TX (US)

(21) Appl. No.: 17/224,811

(22) Filed: Apr. 7, 2021

Related U.S. Application Data
(63) Continuation of application No. PCT/US2019/057724, filed on Oct. 23, 2019.
(60) Provisional application No. 62/749,911, filed on Oct. 24, 2018.

(51) Int. Cl. B01L 3/00; G01N 33/543

(52) U.S. Cl. CPC B01L 3/5023; B01L 3/502707; B01L 3/502753; B01L 2300/0816; B01L 2300/0883; B01L 2300/0877; B01L 2200/0668; G01N 33/54366

(57) ABSTRACT

A method for the isolation of one or more target(s) of interest using fluidic devices with at least one inlet and at least one outlet; a multidirectional fluidic channel between the at least one inlet and the at least one outlet; said multidirectional fluidic channel comprising at least one wall; a substance coating at least a portion of an inside surface of the at least one wall that detects, captures, adsorbs, and/or removes the target(s) of interest from a sample; an eluant for eluting the materials of interest from the fluidic channel; concentrating a target sample from the eluted material(s) of interest and the subsequent analysis and identification of the eluted materials of interest and/or diagnose of a disease based on the target(s) of interest isolated from the sample.

### CROSS-REFERENCE

This application is a continuation of International Application No. PCT/US2019/057724, filed Oct. 23, 2019, which claims the benefit of U.S. Provisional Patent Application No. 62/749,911, filed Oct. 24, 2018, which are incorporated herein by reference in their entirety.

### SUMMARY OF THE INVENTION

The methods provided herein isolate one or more target(s) of interest from flowing matter using fluidic devices that detect, capture, adsorb, and/or remove the target(s) of interest (alternatively called “disease-causing material(s)” or “material(s) of interest”) from biological fluids and produce an eluant for the subsequent diagnosis and treatment of disease based thereon. Devices are provided herein that are effective in and configured to isolate target(s) of interest from flowing matter by detecting, capturing, adsorbing, and/or removing target(s) of interest from biological fluids. The devices may further be configured to evaluate and/or identify the target(s) of interest and/or diagnose a disease based on the target(s) of interest isolated from the biological fluid. The devices may further be configured to provide information about the target(s) of interest to a system for evaluation and/or identification of the target(s) of interest. The target(s) of interest isolated by the device may be sufficiently preserved by the devices and methods herein that the disease-causing material may be transferred to and evaluated by a second system or device by traditional methods known to one of skill in the art.

The detection, capture, adsorption, and/or removal of the disease-causing material from the biological fluids itself may result in a treatment for the disease in a subject and/or reduction or elimination of symptoms of the disease in the subject. The detection, capture, adsorption, and/or removal of the disease-causing material from the biological fluids may provide practitioners with sufficient information to treat a disease or symptoms thereof in a subject, without need for further identification of the disease-causing material. The detection, capture, adsorption, and/or removal of the disease-causing material from the biological fluids may be followed by evaluation of the disease-causing material isolated by and eluted by the device. The evaluation and/or analysis of the eluted materials of interest (or portions thereof or altered versions thereof) may be conducted by the device or by other systems or devices which receive the eluted materials of interest, or a portion thereof, or an altered form thereof, or receive information about the eluted materials of interest. Identification of the disease-causing material or the disease by the device or by another device receiving the disease-causing material, a portion thereof, an altered form of the disease-causing material, or information about the disease-causing material, may be used to reduce or eliminate symptoms of the disease in the subject. In some embodiments, knowledge of a substance coated on the wall of a channel of a device combined with a determination that the substance captured, isolated, adsorbed or otherwise detected from the biological fluid passed through the channel is sufficient to diagnose a disease and/or determine a treatment for the subject, without further evaluation of the material isolated by the device.

The method presented herein is intended to be used for the isolation of targets of interest from flowing matter, and in the diagnosis and treatment of disease. The method presented herein provides a method of capturing and collecting targeted analytes from fluids by flowing said fluids through a multi-directional channel that is coated with a polypeptide antibiotic and/or other materials. However, those familiar with the art would appreciate the broad applicability of this method.

Provided herein is a method for the capture and isolation of materials of interest comprising: flowing a sample through a fluidic cartridge comprising a multi-directional fluidic channel between an inlet and an outlet; adsorbing one or more materials of interest on at least one wall of the multi-directional fluidic channel; washing the multidirectional fluidic cartridge channel with a wash buffer (eluant) to separate the adsorbed materials of interest from a residual sample matter that is not of interest; discarding said wash buffer containing the residual sample matter that is not of interest; flowing an eluant through the fluidic cartridge containing the adsorbed materials of interest on the at least one wall in the multi-directional fluidic channel between the inlet and the outlet, collecting and concentrating the materials of interest in an eluate; and collecting an amount of the eluate containing the one or more concentrated materials of interest eluted from the sample. In some embodiments, the sample comprises an additive before flowing through the fluidic cartridge. In some embodiments the additive comprises one or more of: an EDTA; a K₂EDTA; a heparin compound; a fluoride; an oxalate; a sodium citrate; a sodium polyanethol sulfonate (SPS); an Acid Citrate Dextrose Solution; a distilled H2O; or a saline.

In some embodiments, a substance is coated on the at least one wall of the multi-directional fluidic channel.

In some embodiments, the eluant is an elution buffer. In some embodiments, the eluant is a lysis buffer.

In some embodiments, the eluate containing the concentrated materials of interest is purified by introducing the eluate into a spin column. In some embodiments, the spin column is placed in a centrifuge for centrifugation. During centrifugation, target sample materials are entrapped in the spin column matrix, while other unwanted materials pass through the spin column matrix into the collection vial. The collection vial is discarded. The targeted, isolated sample material entrapped in the spin column matrix is then released from the matrix and collected into a clean collection vial by washing the matrix with ethyl alcohol.

In some embodiments, the collected isolated materials of interest are analyzed. In some embodiments, the collected isolated materials of interest (or portions thereof or altered versions thereof) are analyzed by other systems or devices which receive the eluted materials of interest, or a portion thereof, or an altered form thereof, or receive information about the eluted materials of interest. In some embodiments, identification of the disease-causing material or the disease by the device or by another device receiving the disease-causing material, a portion thereof, an altered form of the disease-causing material, or information about the disease-causing material, is used to reduce or eliminate symptoms of the disease in the subject.

In some embodiments, a substance or a plurality of the substance is coated on at least one inside wall of the multi-directional fluidic channel. In some embodiments, the substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments, one or more substances or a plurality of the substances are coated on at least one wall of the multi-directional fluidic channel, wherein each substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments, the substance comprises an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent.

In some embodiments, the substance coating the channel wall comprises a fixed, covalently-bonded polypeptide antibiotic. In some embodiments, the fixed, covalently-bonded polypeptide antibiotic is polymyxin. In some embodiments, the amount of polymyxin fixed is about 0.5 mM to about 50 mM. In some embodiments, the amount of polymyxin fixed is at least about 0.5 mM. In some embodiments, the amount of polymyxin fixed is at most about 50 mM. In some embodiments, the amount of polymyxin fixed is about 0.5 mM to about 1 mM, about 0.5 mM to about 5 mM, about 0.5 mM to about 10 mM, about 0.5 mM to about 20 mM, about 0.5 mM to about 30 mM, about 0.5 mM to about 40 mM, about 0.5 mM to about 50 mM, about 1 mM to about 5 mM, about 1 mM to about 10 mM, about 1 mM to about 20 mM, about 1 mM to about 30 mM, about 1 mM to about 40 mM, about 1 mM to about 50 mM, about 5 mM to about 10 mM, about 5 mM to about 20 mM, about 5 mM to about 30 mM, about 5 mM to about 40 mM, about 5 mM to about 50 mM, about 10 mM to about 20 mM, about 10 mM to about 30 mM, about 10 mM to about 40 mM, about 10 mM to about 50 mM, about 20 mM to about 30 mM, about 20 mM to about 40 mM, about 20 mM to about 50 mM, about 30 mM to about 40 mM, about 30 mM to about 50 mM, or about 40 mM to about 50 mM. The term “about” when used in reference to the amount of polymyxin, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the fixed, covalently-bonded polypeptide antibiotic is vancomycin. In some embodiments, the amount of vancomycin fixed is about 0.5 mM to about 50 mM. In some embodiments, the amount of vancomycin fixed is at least about 0.5 mM. In some embodiments, the amount of vancomycin fixed is at most about 50 mM. In some embodiments, the amount of vancomycin fixed is about 0.5 mM to about 1 mM, about 0.5 mM to about 5 mM, about 0.5 mM to about 10 mM, about 0.5 mM to about 20 mM, about 0.5 mM to about 30 mM, about 0.5 mM to about 40 mM, about 0.5 mM to about 50 mM, about 1 mM to about 5 mM, about 1 mM to about 10 mM, about 1 mM to about 20 mM, about 1 mM to about 30 mM, about 1 mM to about 40 mM, about 1 mM to about 50 mM, about 5 mM to about 10 mM, about 5 mM to about 20 mM, about 5 mM to about 30 mM, about 5 mM to about 40 mM, about 5 mM to about 50 mM, about 10 mM to about 20 mM, about 10 mM to about 30 mM, about 10 mM to about 40 mM, about 10 mM to about 50 mM, about 20 mM to about 30 mM, about 20 mM to about 40 mM, about 20 mM to about 50 mM, about 30 mM to about 40 mM, about 30 mM to about 50 mM, or about 40 mM to about 50 mM. The term “about” when used in reference to the amount of vancomycin, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the multi-directional fluidic channel is composed of at least one thermoplastic polymer base material that has at least one surface exposed functional group. In some embodiments, the thermoplastic polymer base material comprises: at least one exposed surface exposed wherein the thermoplastic polymer base material comprises at least one exposed surface selected from the functional group consisting of: a carbonyl group, a carboxyl group, an alcohol group, an amino group, a chloride group, a styrene group, an alpha-halogenated acyl group, a benzyl group, and an isocyanic acid groups, and a remaining thermoplastic polymer base material further comprises other polymers or copolymers comprising: a polyvinyl chloride, a polyvinyl acetate, a polyvinyl benzene, a polytetrafluoroethylene, a polyamide, an acrylamide, a polyurethane, a polyethylene, a polyethylene terephthalate, a polydimethylsiloxane, a polyacrylonitrile, a polycarbonate, an acetal plastic, a polyethylene, a polypropylene, or a polymethyl methacrylate. In some embodiments, the thermoplastic polymer base material is polycarbonate.

In some embodiments, the crosslinking agent to be fixed to the base material is polyethylene glycol or a derivative substance in which the amount fixed is about 1 mM to about 50 mM. In some embodiments, the crosslinking agent to be fixed to the base material is polyethylene glycol or a derivative substance in which the amount fixed is at least about 1 mM. In some embodiments, the crosslinking agent to be fixed to the base material is polyethylene glycol or a derivative substance in which the amount fixed is at most about 50 mM. In some embodiments, the crosslinking agent to be fixed to the base material is polyethylene glycol or a derivative substance in which the amount fixed is about 1 mM to about 5 mM, about 1 mM to about 10 mM, about 1 mM to about 20 mM, about 1 mM to about 30 mM, about 1 mM to about 40 mM, about 1 mM to about 50 mM, about 5 mM to about 10 mM, about 5 mM to about 20 mM, about 5 mM to about 30 mM, about 5 mM to about 40 mM, about 5 mM to about 50 mM, about 10 mM to about 20 mM, about 10 mM to about 30 mM, about 10 mM to about 40 mM, about 10 mM to about 50 mM, about 20 mM to about 30 mM, about 20 mM to about 40 mM, about 20 mM to about 50 mM, about 30 mM to about 40 mM, about 30 mM to about 50 mM, or about 40 mM to about 50 mM. The term about, when used in reference to the amount of crosslinking agent, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the fluidic cartridge is disposable.

In some embodiments of the fluidic cartridge, the multidirectional channel has an internal width of about 0.001 mm to about 1,000 mm. In some embodiments of the fluidic cartridge, the multidirectional channel has an internal width of at least about 0.001 mm. In some embodiments of the fluidic cartridge, the multidirectional channel has an internal width of at most about 1,000 mm. In some embodiments of the fluidic cartridge, the multidirectional channel has an internal width of about 0.001 mm to about 0.005 mm, about 0.001 mm to about 0.1 mm, about 0.001 mm to about 0.05 mm, about 0.001 mm to about 1 mm, about 0.001 mm to about 10 mm, about 0.001 mm to about 50 mm, about 0.001 mm to about 100 mm, about 0.001 mm to about 500 mm, about 0.001 mm to about 1,000 mm, about 0.005 mm to about 0.1 mm, about 0.005 mm to about 0.5 mm, about 0.005 mm to about 1 mm, about 0.005 mm to about 10 mm, about 0.005 mm to about 50 mm, about 0.005 mm to about 100 mm, about 0.005 mm to about 500 mm, about 0.005 mm to about 1,000 mm, about 0.01 mm to about 0.5 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.05 mm to about 1 mm, about 0.05 mm to about 10 mm, about 0.05 mm to about 50 mm, about 0.05 mm to about 100 mm, about 0.05 mm to about 500 mm, about 0.05 mm to about 1,000 mm, about 0.1 mm to about 10 mm, about 0.1 mm to about 50 mm, about 0.1 mm to about 100 mm, about 0.1 mm to about 500 mm, about 0.1 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the internal width of the multi-directional channel, in some embodiments means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” , when used in reference to the width of the multi-directional channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm or 0.01 mm of a given value or range.

In some embodiments of the fluidic cartridge, the multidirectional channel has an internal height of about 0.001 mm to about 100 mm. In some embodiments of the fluidic cartridge, the multidirectional channel has an internal height of at least about 0.001 mm. In some embodiments of the fluidic cartridge, the multidirectional channel has an internal height of at most about 100 mm. In some embodiments of the fluidic cartridge, the multidirectional channel has an internal height of about 0.001 mm to about 0.005 mm, about 0.001 mm to about 0.01 mm, about 0.001 mm to about 0.05 mm, about 0.001 mm to about 0.1 mm, about 0.001 mm to about 0.5 mm, about 0.001 mm to about 1 mm, about 0.001 mm to about 10 mm, about 0.001 mm to about 50 mm, about 0.001 mm to about 100 mm, about 0.005 mm to about 0.01 mm, about 0.005 mm to about 0.05 mm, about 0.005 mm to about 0.1 mm, about 0.005 mm to about 0.5 mm, about 0.005 mm to about 1 mm, about 0.005 mm to about 10 mm, about 0.005 mm to about 50 mm, about 0.005 mm to about 100 mm, about 0.01 mm to about 0.05 mm, about 0.01 mm to about 0.1 mm, about 0.01 mm to about 0.5 mm, about 0.01 mm to about 1 mm, about 0.01 mm to about 10 mm, about 0.01 mm to about 50 mm, about 0.01 mm to about 100 mm, about 0.05 mm to about 0.1 mm, about 0.05 mm to about 0.5 mm, about 0.05 mm to about 1 mm, about 0.05 mm to about 10 mm, about 0.05 mm to about 50 mm, about 0.05 mm to about 100 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.5 mm to about 1 mm, about 0.5 mm to about 10 mm, about 0.5 mm to about 50 mm, about 0.5 mm to about 100 mm, about 1 mm to about 10 mm, about 1 mm to about 50 mm, about 1 mm to about 100 mm, about 10 mm to about 50 mm, about 10 mm to about 100 mm, or about 50 mm to about 100 mm. The term “about”, when used in reference to the height of the multidirectional channel, in some embodiments, means 0.001 mm, 0.005 mm, 0.01 mm, 0.05 mm, 0.1 mm, 0.5 mm, 1 mm, 10 mm, or 50 mm. The term “about”, when used in reference to the height of the multi-directional channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” , when used in reference to the height of the multi-directional channel, means within 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm or 0.01 mm of a given value or range.

In some embodiments of the fluidic cartridge, the multidirectional channel has a length of about 0.01 mm to about 10,000 mm between the inlet and the outlet. In some embodiments of the fluidic cartridge, the multidirectional channel has a length of at least about 0.01 mm between the inlet and the outlet. In some embodiments of the fluidic cartridge, the multidirectional channel has a length of at most about 10,000 mm between the inlet and the outlet. In some embodiments of the fluidic cartridge, the multidirectional channel has a length between the inlet and the outlet of about 0.001 mm to about 0.05 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.01 mm to about 5,000 mm, about 0.01 mm to about 10,000 mm, about 0.05 mm to about 1 mm, about 0.05 mm to about 10 mm, about 0.05 mm to about 50 mm, about 0.50 mm to about 100 mm, about 0.05 mm to about 500 mm, about 0.05 mm to about 1,000 mm, about 0.05 mm to about 5,000 mm, about 0.05 mm to about 10,000 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.1 mm to about 500 mm, about 0.1 mm to about 1,000 mm, about 0.1 mm to about 5,000 mm, about 0.1 mm to about 10,000 mm, about 0.5 mm to about 1 mm, about 0.5 mm to about 10 mm, about 0.5 mm to about 50 mm, about 0.5 mm to about 100 mm, about 0.5 mm to about 500 mm, about 0.5 mm to about 1,000 mm, about 0.5 mm to about 5,000 mm, about 0.5 mm to about 10,000 mm, about 1 mm to about 10 mm, about 1 mm to about 50 mm, about 1 mm to about 100 mm, about 1 mm to about 500 mm, about 1 mm to about 1,000 mm, about 1 mm to about 5,000 mm, about 1 mm to about 10,000 mm, about 10 mm to about 50 mm, about 10 mm to about 100 mm, about 10 mm to about 500 mm, about 10 mm to about 1,000 mm, about 10 mm to about 5,000 mm, about 10 mm to about 10,000 mm, about 50 mm to about 100 mm, about 50 mm to about 500 mm, about 50 mm to about 1,000 mm, about 50 mm to about 5,000 mm, about 50 mm to about 10,000 mm, about 100 mm to about 500 mm, about 100 mm to about 1,000 mm, about 100 mm to about 5,000 mm, about 100 mm to about 10,000 mm, about 500 mm to about 1,000 mm, about 500 mm to about 5,000 mm, about 500 mm to about 10,000 mm, about 1,000 mm to about 5,000 mm, about 1,000 mm to about 10,000 mm, or about 5,000 mm to about 10,000 mm. The term “about”, when used in reference to the length of the multi-directional channel between the inlet and the outlet, in some embodiments means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the length of the multi-directional channel, means within 5,000 mm, 1,000 mm, 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments of the fluidic cartridge, the multidirectional channel has a length of about 0.1 mm to about 3,000.0 mm between the inlet and the outlet. In some embodiments of the fluidic cartridge, the multidirectional channel has a length of at least about 0.01 mm between the inlet and the outlet. In some embodiments of the fluidic cartridge, the multidirectional channel has a length of at most about 3,000 mm between the inlet and the outlet. In some embodiments of the fluidic cartridge, the multidirectional channel has a length between the inlet and the outlet of about 0.001 mm to about 0.05 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.01 mm to about 2,000 mm, about 0.01 mm to about 3,000 mm, about 0.05 mm to about 1 mm, about 0.05 mm to about 10 mm, about 0.05 mm to about 50 mm, about 0.50 mm to about 100 mm, about 0.05 mm to about 500 mm, about 0.05 mm to about 1,000 mm, about 0.05 mm to about 2,000 mm, about 0.05 mm to about 3,000 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.1 mm to about 500 mm, about 0.1 mm to about 1,000 mm, about 0.1 mm to about 2,000 mm, about 0.1 mm to about 3,000 mm, about 0.5 mm to about 1 mm, about 0.5 mm to about 10 mm, about 0.5 mm to about 50 mm, about 0.5 mm to about 100 mm, about 0.5 mm to about 500 mm, about 0.5 mm to about 1,000 mm, about 0.5 mm to about 2,000 mm, about 0.5 mm to about 3,000 mm, about 1 mm to about 10 mm, about 1 mm to about 50 mm, about 1 mm to about 100 mm, about 1 mm to about 500 mm, about 1 mm to about 1,000 mm, about 1 mm to about 2,000 mm, about 1 mm to about 3,000 mm, about 10 mm to about 50 mm, about 10 mm to about 100 mm, about 10 mm to about 500 mm, about 10 mm to about 1,000 mm, about 10 mm to about 2,000 mm, about 10 mm to about 3,000 mm, about 50 mm to about 100 mm, about 50 mm to about 500 mm, about 50 mm to about 1,000 mm, about 50 mm to about 2,000 mm, about 50 mm to about 3,000 mm, about 100 mm to about 500 mm, about 100 mm to about 1,000 mm, about 100 mm to about 2,000 mm, about 100 mm to about 3,000 mm, about 500 mm to about 1,000 mm, about 500 mm to about 2,000 mm, about 500 mm to about 3,000 mm, about 1,000 mm to about 2,000 mm, about 1,000 mm to about 3,000 mm, or about 2,000 mm to about 3,000 mm. The term “about”, when used in reference to the length of the multi-directional channel between the inlet and the outlet, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the length of the multi-directional channel, means within 1,500 mm, 1,000 mm, 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments, said multi-directional channel is spiral shaped. In some embodiments of the fluidic cartridge, the spiral shaped channel has a radius of curvature ranging between about 0.01 mm to about 1,000 mm. In some embodiments of the fluidic cartridge, the spiral shaped channel has an inner-most radius of curvature at least about 0.01 mm. In some embodiments of the fluidic cartridge, the spiral shaped channel has an outer-most radius of curvature at most about 1,000 mm. In some embodiments of the fluidic cartridge, the spiral shaped channel has a radius of curvature between about 0.01 mm to about 10 mm, about 0.01 mm to about 50 mm, about 0.01 mm to about 100 mm, about 0.01 mm to about 500 mm, about 0.01 mm to about 1,000 mm, about 0.1 mm to about 10 mm, about 0.1 mm to about 50 mm, about 0.1 mm to about 100 mm, about 0.1 mm to about 500 mm, about 0.1 mm to about 1,000 mm, about 1 mm to about 10 mm, about 1 mm to about 50 mm, about 1 mm to about 100 mm, about 1 mm to about 500 mm, about 1 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the radius of curvature of the spiral shaped channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the outer-most radius of curvature of the spiral shaped channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments, the spiral shaped channel has a radius of curvature ranging between about 5 mm to about 100.0 mm. In some embodiments of the fluidic cartridge, the spiral shaped channel has an inner-most radius of curvature of at least about 5 mm. In some embodiments of the fluidic cartridge, the spiral shaped channel has an outer-most radius of curvature of at most about 100 mm. In some embodiments of the fluidic cartridge, the spiral shaped channel has a radius of curvature between about 5 mm to about 10 mm, about 5 mm to about 25 mm, about 5 mm to about 50 mm, about 5 mm to about 75 mm, about 5 mm to about 100 mm, about 10 mm to about 25 mm, about 10 mm to about 50 mm, about 10 mm to about 75 mm, about 10 mm to about 100 mm, about 25 mm to about 50 mm, about 25 mm to about 75 mm, about 25 mm to about 100 mm, about 50 mm to about 75 mm, about 50 mm to about 100 mm, or about 75 mm to about 100 mm. The term “about”, when used in reference to the radius of curvature of the spiral shaped channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the outer-most radius of curvature of the spiral shaped channel, means within 5.0 mm, 2.5 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments, the spiral shaped multi-directional channel has a distance between channel edges in the spiral of about 0.01 to about 1,000 mm. In some embodiments of the fluidic cartridge, the spiral shaped multidirectional channel has a distance between the channel edges in the spiral of at least about 0.01 mm. In some embodiments of the fluidic cartridge, the spiral shaped multidirectional channel has a distance between the channel edges in the spiral of at most about 1,000 mm. In some embodiments of the fluidic cartridge, the spiral shaped multidirectional channel has a distance between the channels in the spiral about 0.01 mm to about 0.05 mm, about 0.01 mm to about 0.1 mm, about 0.01 mm to about 0.5 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.05 mm to about 0.1 mm, about 0.05 mm to about 0.5 mm, about 0.05 mm to about 1 mm, about 0.05 mm to about 10 mm, about 0.05 mm to about 50 mm, about 0.05 mm to about 100 mm, about 0.05 mm to about 500 mm, about 0.05 mm to about 1,000 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.1 mm to about 500 mm, about 0.1 mm to about 1,000 mm, about 0.5 mm to about 1 mm, about 0.5 mm to about 10 mm, about 0.5 mm to about 50 mm, about 0.5 mm to about 100 mm, about 0.5 mm to about 500 mm, about 0.5 mm to about 1,000 mm, about 1 mm to about 10 mm, about 1 mm to about 50 mm, about 1 mm to about 100 mm, about 1 mm to about 500 mm, about 1 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the distance between the channel edges in the spiral, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the distance between the channels in the spiral, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 2.5 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments, said multi-directional channel is helically shaped. In some embodiments, said multi-directional channel is fabricated around a cylindrical chamber. In some embodiments, said multi-directional channel is helically shaped and fabricated around a cylindrical chamber. In some embodiments of the fluidic cartridge, the helical shaped multidirectional channel has a radius of curvature ranging between about 1 mm to about 1,000 mm. In some embodiments of the fluidic cartridge, the helical shaped multidirectional channel has a radius of curvature of at least about 1 mm. In some embodiments of the fluidic cartridge, the helical shaped multidirectional channel has a radius of curvature of at most about 1,000 mm. In some embodiments of the fluidic cartridge, the helical shaped multidirectional channel has a radius of curvature ranging between about 1 mm to about 5 mm, about 1 mm to about 10 mm, about 1 mm to about 50 mm, about 1 mm to about 100 mm, about 1 mm to about 500 mm, about 1 mm to about 1,000 mm, about 5 mm to about 10 mm, about 5 mm to about 50 mm, about 5 mm to about 100 mm, about 5 mm to about 500 mm, about 5 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the radius of curvature of the helical shaped multidirectional channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the radius of curvature of the helical shaped multidirectional channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 2.5 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments of the fluidic cartridge, the helical shaped multidirectional channel has a pitch between about 1 mm to about 1,000 mm. In some embodiments of the fluidic cartridge, the helical shaped multidirectional channel has a pitch of at least about 1 mm. In some embodiments of the fluidic cartridge, the helical shaped multidirectional channel has a pitch of at most about 1,000 mm. In some embodiments of the fluidic cartridge, the helical shaped multidirectional channel has a pitch between about 1 mm to about 5 mm, about 1 mm to about 10 mm, about 1 mm to about 50 mm, about 1 mm to about 100 mm, about 1 mm to about 500 mm, about 1 mm to about 1,000 mm, about 5 mm to about 10 mm, about 5 mm to about 50 mm, about 5 mm to about 100 mm, about 5 mm to about 500 mm, about 5 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the pitch of the helical shaped multidirectional channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the pitch of the helical shaped multidirectional channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 2.5 mm, 1.0 mm, 0.9 mm, 0.8 mm, 0.7 mm, 0.6 mm, 0.5 mm, 0.4 mm, 0.3 mm, 0.2 mm or 0.1 mm of a given value or range.

In some embodiments, the multi-directional channel is enclosed. In some embodiments, the multi-directional channel is enclosed between the at least one inlet and the at least one outlet. In some embodiments, the multi-directional channel is enclosed using at least one of: bolts, an adhesive, a binding material, a resin, an inner sleeve and an outer sleeve; utilizing at least one of: a base plate, a cover glass, a curing (method), a thermal expansion (process), ultrasonic welding, vibration welding, high frequency welding—(aka: radio frequency welding, and dielectric welding), heated tool or plate welding, solvent bonding, laser welding, spin welding, infrared welding, and adhesive bonding.

In some embodiments, the fluidic cartridge comprising the multi-directional channel is fabricated using at least one method selected from the group consisting of: 3-D printing, stereolithography, photolithography, injection molding, blow molding, casting, ultrasonic welding, vibration welding, high frequency welding—(aka: radio frequency welding, and dielectric welding), heated tool or plate welding, solvent bonding, laser welding, spin welding, infrared welding, adhesive bonding, machining, turning, drilling, boring, reaming, electric discharge machining (EDM) and milling.

In some embodiments of the method, syringes are attached to the at least one multi-directional channel inlet or at least one channel outlet with: fittings, caps or luer lock connectors.

In some embodiments of the method, the syringe is a luer lock syringe. In some embodiments of the method, a fluid sample is brought in contact with a multidirectional, polypeptide antibiotic coated channel using a pump. In some embodiments of the method, following adsorbing said material(s) of interest on said multidirectional channel walls, an elution/lysis buffer is brought in contact with a multidirectional, polypeptide antibiotic coated channel using a syringe. In some embodiments, the syringe is a luer lock syringe.

In some embodiments of the method the elution/lysis buffer, (alternatively called the eluant, herein) is selected from a group comprising: a Tris-HCl; an Ethylenediaminetetraacetic acid (EDTA); a sodium dodecyl sulfate (SDS); a TritonX-100; a chaotropic buffer; or a proteinase K; sodium heparin; a heparin compound; fluoride; oxalate; sodium citrate; sodium polyanethol sulfonate (SPS); Acid Citrate Dextrose Solution (sometimes called Anticoagulant Citrate Dextrose Solution); distilled H₂O; or saline.

In some embodiments of the method, the eluant is selected from a group comprising: Tris-HCl, Ethylenediaminetetraacetic acid (EDTA), sodium dodecyl sulfate (SDS), TritonX-100, a chaotropic buffer or proteinase K. In some embodiments of the method, the eluant contains between 0.001M to about 100 M of Tris-HCL. In some embodiments of the method, the eluant contains between 0.001M to about 100M of EDTA. In some embodiments, the eluant has a pH between a range of 2 and 13. In preferred embodiments the eluant has a pH between a range of 6 and 9.

In some embodiments of the method, the eluant contains between about 0.001 M to about 100 M. In some embodiments of the method, the eluant contains between at least about 0.001 M. In some embodiments of the method, the eluant contains between at most about 100 M. In some embodiments of the method, the eluant contains between about 0.001 M to about 0.01 M, about 0.001 M to about 0.1 M, about 0.001 M to about 1 M, about 0.001 M to about 10 M, about 0.001 M to about 25 M, about 0.001 M to about 50 M, about 0.001 M to about 75 M, about 0.001 M to about 100 M, about 0.01 M to about 0.1 M, about 0.01 M to about 1 M, about 0.01 M to about 10 M, about 0.01 M to about 25 M, about 0.01 M to about 50 M, about 0.01 M to about 75 M, about 0.01 M to about 100 M, about 0.1 M to about 1 M, about 0.1 M to about 10 M, about 0.1 M to about 25 M, about 0.1 M to about 50 M, about 0.1 M to about 75 M, about 0.1 M to about 100 M, about 1 M to about 10 M, about 1 M to about 25 M, about 1 M to about 50 M, about 1 M to about 75 M, about 1 M to about 100 M, about 10 M to about 25 M, about 10 M to about 50 M, about 10 M to about 75 M, about 10 M to about 100 M, about 25 M to about 50 M, about 25 M to about 75 M, about 25 M to about 100 M, about 50 M to about 75 M, about 50 M to about 100 M, or about 75 M to about 100 M. The term “about” when used in reference to the amount of eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the eluant has a pH of about 2 to about 13. In some embodiments, the eluant has a pH of at least about 2. In some embodiments, the eluant has a pH of at most about 13. In some embodiments, the eluant has a pH of about 2 to about 3, about 2 to about 4, about 2 to about 5, about 2 to about 6, about 2 to about 7, about 2 to about 8, about 2 to about 9, about 2 to about 10, about 2 to about 11, about 2 to about 12, about 2 to about 13, about 3 to about 4, about 3 to about 5, about 3 to about 6, about 3 to about 7, about 3 to about 8, about 3 to about 9, about 3 to about 10, about 3 to about 11, about 3 to about 12, about 3 to about 13, about 4 to about 5, about 4 to about 6, about 4 to about 7, about 4 to about 8, about 4 to about 9, about 4 to about 10, about 4 to about 11, about 4 to about 12, about 4 to about 13, about 5 to about 6, about 5 to about 7, about 5 to about 8, about 5 to about 9, about 5 to about 10, about 5 to about 11, about 5 to about 12, about 5 to about 13, about 6 to about 7, about 6 to about 8, about 6 to about 9, about 6 to about 10, about 6 to about 11, about 6 to about 12, about 6 to about 13, about 7 to about 8, about 7 to about 9, about 7 to about 10, about 7 to about 11, about 7 to about 12, about 7 to about 13, about 8 to about 9, about 8 to about 10, about 8 to about 11, about 8 to about 12, about 8 to about 13, about 9 to about 10, about 9 to about 11, about 9 to about 12, about 9 to about 13, about 10 to about 11, about 10 to about 12, about 10 to about 13, about 11 to about 12, about 11 to about 13, or about 12 to about 13.

In some preferred embodiments, the eluant has a pH of about 6 to about 9. In some preferred embodiments, the eluant has a pH of at least about 6. In some preferred embodiments, the eluant has a pH of at most about 9. In some preferred embodiments, the eluant has a pH of about 6 to about 7, about 6 to about 8, about 6 to about 9, about 7 to about 8, about 7 to about 9, or about 8 to about 9.

Provided herein is a method for isolation of materials of interest comprising: bringing a sample of fluid in contact with a multidirectional, polypeptide antibiotic coated channel; adsorbing (capturing) said material(s) of interest on said multidirectional channel walls; eluting and lysing said captured materials of interest with an elution/lysis buffer; and analyzing the presence or amount of said materials of interest eluted from the multidirectional, polypeptide antibiotic coated channel.

In some embodiments of the method, a fluid sample is brought in contact with a multidirectional, polypeptide antibiotic coated channel using a syringe. In some embodiments of the method, the syringe is a luer lock syringe. In some embodiments of the method, a fluid sample is brought in contact with a multidirectional, polypeptide antibiotic coated channel using a pump. In some embodiments of the method, following adsorbing said material(s) of interest on said multidirectional channel walls, an elution/lysis buffer is brought in contact with a multidirectional, polypeptide antibiotic coated channel using a syringe. In some embodiments, the syringe is a luer lock syringe.

In some embodiments of the method the elution/lysis buffer, (alternatively called the eluant, herein) is selected from a group comprising: a Tris-HCl; an Ethylenediaminetetraacetic acid (EDTA); a sodium dodecyl sulfate (SDS); a TritonX-100; a chaotropic buffer; or a proteinase K; sodium heparin; a heparin compound; fluoride; oxalate; sodium citrate; sodium polyanethol sulfonate (SPS); Acid Citrate Dextrose Solution (sometimes called Anticoagulant Citrate Dextrose Solution); distilled H₂O; or saline.

In some embodiments of the method, the eluant is selected from a group comprising: Tris-HCl, Ethylenediaminetetraacetic acid (EDTA), sodium dodecyl sulfate (SDS), TritonX-100, a chaotropic buffer or proteinase K. In some embodiments of the method, the eluant contains between 0.001M to about 100 M of Tris-HCL. In some embodiments of the method, the eluant contains between 0.001M to about 100M of EDTA. In some embodiments, the eluant has a pH between a range of 2 and 13. In preferred embodiments the eluant has a pH between a range of 6 and 9. In some embodiments of the method, the elution buffer contains between 0.01% and about 90% of sodium dodecyl sulfate having a pH between 6 and 9. In some embodiments of the method, the elution buffer contains between 0.01% and about 90% of TritonX-100 having a pH between 6 and 9. In some embodiments of the method, the elution buffer contains between 0.001M and about 100M of a chaotropic buffer having a pH between 6 and 9. In some embodiments of the method, the elution buffer contains the chaotropic buffer Guanidinium thiocyanate having a pH between 6 and 9. In some embodiments of the method, the elution buffer contains between 0.001 mg/mL and about 100 mg/mL of proteinase K having a pH between 6 and 9.

In some embodiments of the method, the eluant contains between about 0.001 M to about 100 M. In some embodiments of the method, the eluant contains between at least about 0.001 M. In some embodiments of the method, the eluant contains between at most about 100 M. In some embodiments of the method, the eluant contains between about 0.001 M to about 0.01 M, about 0.001 M to about 0.1 M, about 0.001 M to about 1 M, about 0.001 M to about 10 M, about 0.001 M to about 25 M, about 0.001 M to about 50 M, about 0.001 M to about 75 M, about 0.001 M to about 100 M, about 0.01 M to about 0.1 M, about 0.01 M to about 1 M, about 0.01 M to about 10 M, about 0.01 M to about 25 M, about 0.01 M to about 50 M, about 0.01 M to about 75 M, about 0.01 M to about 100 M, about 0.1 M to about 1 M, about 0.1 M to about 10 M, about 0.1 M to about 25 M, about 0.1 M to about 50 M, about 0.1 M to about 75 M, about 0.1 M to about 100 M, about 1 M to about 10 M, about 1 M to about 25 M, about 1 M to about 50 M, about 1 M to about 75 M, about 1 M to about 100 M, about 10 M to about 25 M, about 10 M to about 50 M, about 10 M to about 75 M, about 10 M to about 100 M, about 25 M to about 50 M, about 25 M to about 75 M, about 25 M to about 100 M, about 50 M to about 75 M, about 50 M to about 100 M, or about 75 M to about 100 M. The term “about” when used in reference to the amount of eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the eluant has a pH of about 2 to about 13. In some embodiments, the eluant has a pH of at least about 2. In some embodiments, the eluant has a pH of at most about 13. In some embodiments, the eluant has a pH of about 2 to about 3, about 2 to about 4, about 2 to about 5, about 2 to about 6, about 2 to about 7, about 2 to about 8, about 2 to about 9, about 2 to about 10, about 2 to about 11, about 2 to about 12, about 2 to about 13, about 3 to about 4, about 3 to about 5, about 3 to about 6, about 3 to about 7, about 3 to about 8, about 3 to about 9, about 3 to about 10, about 3 to about 11, about 3 to about 12, about 3 to about 13, about 4 to about 5, about 4 to about 6, about 4 to about 7, about 4 to about 8, about 4 to about 9, about 4 to about 10, about 4 to about 11, about 4 to about 12, about 4 to about 13, about 5 to about 6, about 5 to about 7, about 5 to about 8, about 5 to about 9, about 5 to about 10, about 5 to about 11, about 5 to about 12, about 5 to about 13, about 6 to about 7, about 6 to about 8, about 6 to about 9, about 6 to about 10, about 6 to about 11, about 6 to about 12, about 6 to about 13, about 7 to about 8, about 7 to about 9, about 7 to about 10, about 7 to about 11, about 7 to about 12, about 7 to about 13, about 8 to about 9, about 8 to about 10, about 8 to about 11, about 8 to about 12, about 8 to about 13, about 9 to about 10, about 9 to about 11, about 9 to about 12, about 9 to about 13, about 10 to about 11, about 10 to about 12, about 10 to about 13, about 11 to about 12, about 11 to about 13, or about 12 to about 13.

In some preferred embodiments, the eluant has a pH of about 6 to about 9. In some preferred embodiments, the eluant has a pH of at least about 6. In some preferred embodiments, the eluant has a pH of at most about 9. In some preferred embodiments, the eluant has a pH of about 6 to about 7, about 6 to about 8, about 6 to about 9, about 7 to about 8, about 7 to about 9, or about 8 to about 9.

In some embodiments of the method, the eluted material of interest, alternatively called, the “eluted target(s) of interest,” “eluted disease-causing material(s),” “eluted materials of interest,” or “eluted disease material,” used synonymously herein, is separated from the eluant, concentrated and washed. In some embodiments of the method, the eluant of interest retained from the fluidic cartridge outlet is washed using spin filtration.

In some embodiments of the method, the elution buffer lyses and breaks open the pathogens, thereby killing them, leaving them suspended in the eluate. In some embodiments of the method, the elution buffer breaks the bonds between the pathogens and colistin/vancomycin, thereby leaving the pathogens alive and intact in the eluate and available for culturing.

In some embodiments of the method, detection of the presence or amount of materials of interest captured within the multidirectional, polypeptide antibiotic coated channel is performed by analyzing the materials of interest following elution from the fluidic cartridge using said elution/lysis buffer. In some embodiments, an analysis method is selected from the group comprising: cell counting, MALDI-TOF MS (matrix assisted laser desorption ionization-time of flight mass spectrometry), mass spectrometry, PCR (polymerase chain reaction), biosensing, flow cytometry, and fluorescent labeling. In some embodiments, the eluted material of interest is analyzed using at least one of: a polymerase chain reaction (PCR), a matrix-assisted laser desorption/ionization-time of flight, (MALDI-TOF), a nuclear magnetic resonance (NMR) spectroscopy, culturing, a fluorescence in situ hybridization (FISH), optically active microbeads and optically active nanoparticles.

Provided herein is a method of isolating one or more target(s) of interest from a sample of flowing matter using fluidic devices that detect, capture, adsorb, and/or remove the target(s) of interest (alternatively called “disease-causing material(s)” or “material(s) of interest”) from biological fluids and the subsequent diagnosis and treatment of disease based thereon. The method comprises; (1) flowing the sample through a multidirectional fluidic cartridge channel that is coated with a substance, causing the sample to come in contact with the coating substance, thereby forming a bond between said substance and said the materials of interest within the sample; (2) separating the bound materials of interest from sample matter that is not of interest by washing the multidirectional fluidic cartridge channel with a wash buffer and then discarding said wash buffer containing the residual sample matter that is not of interest; (3) collecting the materials of interest by flowing an elution buffer (eluant) through the multidirectional fluidic cartridge channel, thus concentrating the materials of interest in an eluate; and (4) the eluate containing the concentrated materials of interest is purified by introducing the eluate into a spin column. The spin column is placed in a centrifuge for centrifugation. During centrifugation, target sample materials are entrapped in the spin column matrix, while other unwanted materials pass through the spin column matrix into the collection vial. The collection vial is discarded. The targeted, isolated sample material entrapped in the spin column matrix is then released from the matrix and collected into a clean collection vial by washing the matrix with ethyl alcohol. In some embodiments, the collected isolated materials of interest are analyzed. Analysis of the isolated materials of interest comprises a polymerase chain reaction (PCR)-based assay, a nuclear magnetic resonance (NMR) spectroscopy, a matrix-assisted laser desorption/ionization-time of flight (MALDI-TOF), a fluorescence in situ hybridization (FISH), an immunoassay, an enzyme-linked immunosorbent assay (ELISA), optically active microbeads, optically active nanoparticles, a culturing assay with optional staining, or a combination thereof.

In some embodiments, the sample is selected from the group consisting of a blood, a serum, a plasma, a urine, a cerebral spinal fluid, a bronchial fluid, another bodily fluid, a homogenized tissue, a homogenized food sample, and a combination thereof.

In some embodiments, the wash buffer comprises a saline, an ethanol, a sterile H₂O, or a combination thereof.

Provided herein is a kit for the capture and adsorption of materials of interest comprising: a fluidic cartridge with an inlet and an outlet; a multidirectional fluidic channel between the inlet and the outlet; said multidirectional fluidic channel comprising: at least one wall; a substance coating an inside wall of the multidirectional fluidic channel; and an eluant for eluting the at least one material of interest from the multidirectional fluidic channel.

In some embodiments of the kit, the substance is coating at least a portion of the inside of the at least one wall of the multi-directional fluidic channel. In some embodiments of the kit, the substance is configured to adsorb one or more materials of interest. In some embodiments of the kit, the eluant is an elution buffer. In some embodiments of the kit, the eluant is a lysis buffer. In some embodiments of the kit, the substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments of the kit, one or more substances or a plurality of the substances are coated on at least one wall of the multi-directional fluidic channel, wherein each substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments of the kit, the substance coating the channel wall comprises a fixed, covalently-bonded polypeptide antibiotic. In some embodiments of the kit, the fixed, covalently-bonded polypeptide antibiotic is polymyxin. In some embodiments of the kit, an amount of polymyxin fixed is at least 0.5 mM. In some embodiments of the kit, the amount of polymyxin fixed is about 1.0 to about 50.0 mM. In some embodiments of the kit, the fixed, covalently-bonded polypeptide antibiotic is vancomycin. In some embodiments of the kit, an amount of vancomycin fixed is at least 0.5 mM. In some embodiments of the kit, the amount of vancomycin fixed is about 1.0 to about 50.0 mM. In some embodiments of the kit, the multi-directional fluidic channel is composed of at least one thermoplastic polymer base material having at least one surface-exposed functional group. In some embodiments of the kit, the thermoplastic polymer base material comprises at least one exposed surface selected from the functional group consisting of: a carbonyl group, a carboxyl group, an alcohol group, an amino group, a chloride group, a styrene group, an alpha-halogenated acyl group, a benzyl group, and an isocyanic acid group, and a remaining thermoplastic polymer base material further comprises other polymers or copolymers. In some embodiments of the kit, the remaining thermoplastic polymer base materials comprise: a polyvinyl chloride; a polyvinyl acetate; a polyvinyl benzene; a polytetrafluoroethylene; a polyamide; an acrylamide; a polyurethane; a polyethylene; a polyethylene terephthalate; a polydimethylsiloxane; a polyacrylonitrile; a polycarbonate; an acetal; a polyethylene; a polypropylene; or a polymethyl methacrylate. In some embodiments of the kit, the thermoplastic polymer base material is polycarbonate. In some embodiments of the kit, the thermoplastic polymer base material is polymethyl methacrylate (PMMA). In some embodiments of the kit, the fluidic cartridge is disposable. In some embodiments of the kit, a crosslinking agent to be fixed to the base material is polyethylene glycol or a derivative substance. In some embodiments of the kit, an amount of the crosslinking agent to be fixed is about 1 mM to about 50 mM. In some embodiments of the kit, an amount of the crosslinking agent to be fixed is at least about 1 mM. In some embodiments of the kit, an amount of the crosslinking agent to be fixed is at most about 50 mM.

### INCORPORATION BY REFERENCE

All publications, patents, and patent applications mentioned in this specification are herein incorporated by reference to the same extent as if each individual publication, patent, or patent application was specifically and individually indicated to be incorporated by reference. This application expressly incorporates herein by reference the contents of PCT Application No. PCT/US17/41038.

### BRIEF DESCRIPTION OF THE DRAWINGS

The novel features of the method are set forth with particularity in the appended claims. A better understanding of the features and advantages of the present disclosure will be obtained by reference to the following detailed description that sets forth illustrative embodiments, in which the principles of the various embodiments of the disclosure are utilized, and the accompanying drawings of which:

FIG. 1 illustrates a representation of particles flowing through a channel and associate force, in accordance with some embodiments;

FIG. 2 illustrates a sample of whole blood infected with bacteria being moved through a double-spiral fluidic cartridge using a syringe, in accordance with some embodiments;

FIG. 3 illustrates the chemistry of functionalization of polycarbonate surface with polymyxin (i.e. colistin), in accordance with some embodiments;

FIG. 4 illustrates the chemistry of functionalization of polycarbonate surface with vancomycin, in accordance with some embodiments;

FIG. 5 illustrates chemistry of functionalization of polycarbonate surface with a crosslinking agent and polymyxin E (i.e. colistin), in accordance with some embodiments;

FIG. 6 illustrates chemistry of functionalization of polycarbonate surface with a crosslinking agent and, in accordance with some embodiments;

FIG. 7A and FIG. 7B illustrate a non-limiting embodiment of a spiral-shaped multidirectional channel, in accordance with some embodiments;

FIG. 8A and FIG. 8B illustrate another non-limiting embodiment of a spiral-shaped multidirectional channel connected to two syringes using luer lock fittings, in accordance with some embodiments;

FIG. 9A and FIG. 9B illustrate another example of a multidirectional channel using a two-part construction and connected to a reservoir, in accordance with some embodiments;

FIG. 10A, FIG. 10B, and FIG. 10C illustrate three views of a top plate of the device of FIG. 9A and FIG. 9B, in accordance with some embodiments;

FIG. 11A, FIG. 11B, FIG. 11C, and FIG. 11D illustrate four views of a bottom plate of the device of FIG. 9A and FIG. 9B, in accordance with some embodiments;

FIG. 12A, FIG. 12B, and FIG. 12C illustrate three views of a reservoir of the device of FIG. 9A and FIG. 9B, in accordance with some embodiments;

FIG. 13A, FIG. 13B, and FIG. 13C illustrate three views of a valve of the device of FIG. 9A and FIG. 9B, in accordance with some embodiments;

FIG. 14 illustrates another example of a multidirectional channel which may use gravity flow, in accordance with some embodiments;

FIGS. 15A-15I illustrate a method of isolating one or more target(s) of interest from a sample of flowing matter, in accordance with some embodiments; and

FIG. 16 is an example of rapid Acinetobacter baumannii detection based on real-time qPCR.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION OF THE INVENTION

Provided herein is a method for the capture and isolation of materials of interest comprising: flowing a sample through a fluidic cartridge comprising a multi-directional fluidic channel between an inlet and an outlet; adsorbing one or more materials of interest on at least one wall of the multi-directional fluidic channel; washing the multidirectional fluidic cartridge channel with a wash buffer (eluant) to separate the adsorbed materials of interest from a residual sample matter that is not of interest; discarding said wash buffer containing the residual sample matter that is not of interest; flowing an eluant through the fluidic cartridge containing the adsorbed materials of interest on the at least one wall in the multi-directional fluidic channel between the inlet and the outlet, collecting and concentrating the materials of interest in an eluate; and collecting an amount of the eluate containing the one or more concentrated materials of interest eluted from the sample.

Provided herein is a kit for the capture and adsorption of materials of interest comprising: a fluidic cartridge with an inlet and an outlet; a multidirectional fluidic channel between the inlet and the outlet; said multidirectional fluidic channel comprising at least one wall; a substance coating an inside wall of the multidirectional fluidic channel; and an eluant for eluting the at least one material of interest from the multidirectional fluidic channel. In some embodiments, the eluant is an elution buffer. In some embodiments, the eluant is a lysis buffer.

The presence of bacteria in the circulatory blood initiates a cascade of local and systemic regulatory mechanisms that often result in sepsis. Sepsis afflicts over 1.5 million Americans annually and has an associated mortality rate ranging from 25-50%. Sepsis is the leading cause of death of the critically ill in the United States, costing the U.S. over $24 Billion in treatment annually. With mortality rates as high as 50% for certain bloodstream infections and fewer antibiotics in development, there exists a tremendous clinical need to address this growing global health problem of sepsis.

A diagnosis of a septic episode is made from blood cultures. Blood cultures consist of a blood culture bottle inoculated with blood from the patient and incubated. It typically takes between 8-36 hours for a blood culture vial to be flagged as positive. The positive flag is followed by identification testing and typically requires two or more additional days. Treatment of patients using the correct antibiotic regime improves patient outcomes, decreases mortality rates, decreases length of hospital stay, and lowers hospital costs. The method herein describes a fluidic cartridge capable of expediting organism detection and identification directly from blood through reduction and/or elimination of the need for bacterial culturing.

Polymyxin, also known as colistin, is a cationic polypeptide antibiotic used for the treatment of Gram-negative infection and for detoxifying endotoxin. The positive charge of polymyxin, or colistin, allows for binding to the negatively charged outer membrane of Gram-negative pathogens and endotoxin. Although it is a powerful antibiotic, its use is limited due to its nephro- and neuro-toxicity. Therefore, its intravenous use is limited. Vancomycin is a polypeptide antibiotic that is commonly used for the treatment of Gram-positive infections. Vancomycin interacts with the cell wall of Gram-positive pathogens and inhibits bacterial cell wall assembly. This leads to activation of bacterial autolysins that destroy the cell wall by lysis. However, vancomycin is nephrotoxic when administered systemically. It is known that polymyxin, immobilized on beads, is capable of absorbing endotoxin. However, the use of beads, nanoparticles, porous materials, and fibers have proven inefficient in the capture of endotoxin from fluids such as blood. Also, none of these technologies are capable of efficient capture and adsorption of blood-borne Gram-positive and Gram-negative pathogens, both of which are root causes of sepsis.

As used herein, and unless otherwise specified, the phrase “material of interest”, “target(s) of interest,” “target material of interest”, “disease-causing material(s),” “materials of interest,” or “disease material,” used synonymously herein, means one or more disease materials comprising cancer cells, circulating tumor cells, peptides, beta amyloid, proteins, enzymes, toxins, diseased cells, infectious microorganisms, cells, parasites, fungi, viruses, microorganisms, bacteria, bacterial toxin, quorum sensing proteins or receptors, lipopolysaccharide, cytokines, IL-Iβ, IL-4, IL-6, IL-8, IL-10, IL-11, IL-13, IL-15, IL-16, tumor necrosis factors, procalcitonin, pathogen-associated molecular patterns, C reactive protein, or a small or protein bound biological molecule relevant to liver failure.

As used herein, and unless otherwise specified, the term “eluant” or “eluent” means a fluid used to elute a substance. Alternatively, an “eluant” or “eluent” is a substance used as a solvent in separating materials in elution. The eluant or eluent is the “carrier” portion of the mobile phase. For example, it moves the analytes through the chromatograph. In liquid chromatography, the eluent is the liquid solvent; in gas chromatography, it is the carrier gas. As used herein, and unless otherwise specified, the term “elute”, “eluting”, “elution”, and like terms, means to wash out or extract, or the process of washing out or extracting; or more specifically to remove (an adsorbed substance) by washing with a solvent. In analytical and organic chemistry, elution is the process of extracting one material from another by washing with a solvent; as in washing of loaded ion-exchange resins to remove captured ions. Further, as used herein, and unless otherwise specified, the term “eluate” means a solution obtained by elution. Still further, as used herein the term “eluate” means the solute, “eluted material of interest”, alternatively called the “eluted target(s) of interest,” “eluted disease-causing material(s),” “eluted materials of interest,” or “eluted disease material,” obtained from the elution process.

It specifically includes both the analytes and solutes passing through the column, while the eluent is only the carrier.

As used herein, and unless otherwise specified, the term “lysis”, “lysing” and similar terms, means the disintegration of a cell by rupture of the cell wall or membrane.

As used herein, and unless otherwise specified, the term “buffer”, “buffering”, pH buffer, hydrogen ion buffer, and similar terms, refers to a solution, or an aqueous solution consisting of a mixture of a weak acid and its conjugate base, or vice versa, that can resist pH change upon the addition of an acidic or basic component. It is able to neutralize small amounts of added acid or base, thus maintaining the pH of the solution relatively stable.

As used herein, and unless otherwise specified, the term “biosensing”, “biosensor” and similar terms, refer to the use of nanoscale or microscale biological sensors. A biosensor is an analytical device, used for the detection of an analyte that combines a biological component with a physicochemical detector. The sensitive biological element, (e.g. tissue, microorganisms, organelles, cell receptors, enzymes, antibodies, nucleic acids, etc.), is a biologically derived material or biomimetic component that interacts, binds, or recognizes with the analyte under study. The biologically sensitive elements can also be created by biological engineering. In a biosensor, the bioreceptor is designed to interact with the specific analyte of interest to produce an effect measurable by the transducer. High selectivity for the analyte among a matrix of other chemical or biological components is a key requirement of the bioreceptor. While the type of biomolecule used can vary widely, biosensors can be classified according to common types of bioreceptor interactions involving: antibody/antigen, enzymes/ligands, nucleic acids/DNA, cellular structures/cells, or biomimetic materials. The bioreceptor is a biomolecule that recognizes the target analyte whereas the transducer converts the recognition event into a measurable signal. The uniqueness of a biosensor is that the two components are integrated into one single sensor.

In some embodiments, the device is configurable be used as a biosensor by adding a fluorescent label to the bacteria, colistin/vancomycin or other endotoxin adsorbing agent, with the fluorescence being activated or increased with target binding to the channel wall. As used herein, the colistin and vancomycin are defined as “ligands”. As used herein, a ligand is a substance that forms a complex with a biomolecule to serve a biological purpose. In protein-ligand binding, the ligand is usually a molecule which produces a signal by binding to a site on a target protein. The fluorescence of the channel is then read directly using a fluorescence detector.

As used herein, and unless otherwise specified, the term “about” or “approximately” means an acceptable error for a particular value as determined by one of ordinary skill in the art, which depends in part on how the value is measured or determined.

As used herein, and unless otherwise specified, in certain embodiments, the term “about” or “approximately” means within 1, 2, 3, or 4 standard deviations. In certain embodiments, the term “about” or “approximately” means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range.

As used herein, and unless otherwise specified, in certain embodiments, the term “about” or “approximately” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

As used herein, and unless otherwise specified, in certain embodiments, the term “about” or “approximately”, means within 1,500 mm, 1,000 mm, 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

As used herein, and unless otherwise specified, in certain embodiments, the term “about” or “approximately” means within 20.0 degrees, 15.0 degrees, 10.0 degrees, 9.0 degrees, 8.0 degrees, 7.0 degrees, 6.0 degrees, 5.0 degrees, 4.0 degrees, 3.0 degrees, 2.0 degrees, 1.0 degrees, 0.9 degrees, 0.8 degrees, 0.7 degrees, 0.6 degrees, 0.5 degrees, 0.4 degrees, 0.3 degrees, 0.2 degrees, 0.1 degrees, 0.09 degrees. 0.08 degrees, 0.07 degrees, 0.06 degrees, 0.05 degrees, 0.04 degrees, 0.03 degrees, 0.02 degrees or 0.01 degrees of a given value or range.

As used herein, and unless otherwise specified, the terms “comprises”, “comprising”, or any other variation thereof, are intended to cover a nonexclusive inclusion, such that a process, method, article, or apparatus that comprises a list of elements does not include only those elements but may include other elements not expressly listed or inherent to such process, method, article, or apparatus.

As used herein, and unless otherwise specified, the terms “user”, “subject” or “patient” are used interchangeably. As used herein, the terms “subject” and “subjects” refers to an animal (e.g., birds, reptiles, and mammals), preferably a mammal including a primate (e.g., a monkey, chimpanzee, and a human) and a non-primate (e.g., a camel, donkey, zebra, cow, pig, horse, cat, dog, rat, and mouse). In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100.

As used herein, and unless otherwise specified, the term “transverse force”, (also called the Euler force) is the tangential force that is felt in reaction to any angular acceleration, also known as azimuthal acceleration or transverse acceleration. It is an acceleration that appears when a non-uniformly rotating reference frame is used for analysis of motion and there is variation in the angular velocity of the reference frame's axes. This definition is typically restricted to a reference frame that rotates about a fixed axis. The Euler force (F) is related to the Euler acceleration by “F=ma”, where “a” is the Euler acceleration and m is the mass of the body. Said another way, the Euler force will be felt by a person riding a merry-go-round. As the ride starts, the Euler force will be the apparent force pushing the person to the back of the horse, and as the ride comes to a stop, it will be the apparent force pushing the person towards the front of the horse. A person on a horse close to the perimeter of the merry-go-round will perceive a greater apparent force than a person on a horse closer to the axis of rotation. The Euler force is perpendicular to the centrifugal force and is in the plane of rotation.

As used herein, and unless otherwise specified, the terms “therapies” and “therapy” can refer to any protocol(s), method(s), compositions, formulations, and/or agent(s) that can be used in the prevention, treatment, management, or amelioration of a condition (e.g., a virus infection or a condition or symptom associated therewith, an infection other than a virus infection or a condition or symptom associated therewith, an IFN treatable disease or a condition in which the attenuated viruses can be used as a vector to induce an immune response to a particular antigen associated with the condition). In certain embodiments, the terms “therapies” and “therapy” refer to biological therapy, supportive therapy, and/or other therapies useful in treatment, management, prevention, or amelioration of a virus infection or a condition or symptom associated therewith, an infection other than a virus infection or a condition or symptom associated therewith, an IFN treatable disease or a condition in which the attenuated viruses can be used as a vector to induce an immune response to a particular antigen associated with the condition, known to one of skill in the art.

As used herein, and unless otherwise specified, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, and unless otherwise specified, the terms “prevent”, “preventing” and “prevention” refer to the inhibition of the development or onset of a condition (e.g.: a virus infection or a condition associated therewith, an infection other than a virus infection or a condition associated therewith, an IFN-treatable disease or a condition in which the attenuated viruses can be used as a vector to induce an immune response to a particular antigen associated with the condition), or the prevention of the recurrence, onset, or development of one or more symptoms of a condition (e.g., virus infection or a condition associated therewith, an infection other than a virus infection or a condition associated therewith, or an IFN-treatable disease), in a subject resulting from the administration of a therapy (e.g., a prophylactic or therapeutic agent), or the administration of a combination of therapies (e.g., a combination of prophylactic or therapeutic agents).

As used herein, and unless otherwise specified, the terms “treat,” “treatment,” and “treating” refer to the eradication or control of virus replication or the replication of a pathogen (e.g., a virus, a bacteria) other than virus, the reduction in the titer of virus or titer other than virus, the reduction in the numbers of a pathogen, the reduction or amelioration of the progression, severity, and/or duration of a condition (e.g., a virus infection or a condition associated therewith, an infection other than a virus infection or a condition or symptom associated therewith, an IFN treatable disease or a condition in which the attenuated viruses can be used as a vector to induce an immune response to a particular antigen associated with the condition), or the amelioration of one or more symptoms resulting from the administration of one or more therapies (including, but not limited to, the administration of one or more prophylactic or therapeutic agents).

As used herein, and unless otherwise specified, the terms “therapeutic agent” and “therapeutic agents” refer to any agent(s) which can be used in the prevention, treatment, management, or amelioration of a condition or a symptom thereof (e.g., an infection or a condition or symptoms associated therewith, an infection other than a virus infection or a condition or symptom associated therewith, an IFN treatable disease or a condition in which the attenuated viruses can be used as a vector to induce an immune response to a particular antigen associated with the condition). Preferably, a therapeutic agent is an agent which is known to be useful for, or has been or is currently being used for the prevention, treatment, management, or amelioration of a virus infection or a condition or symptoms associated therewith, an infection other than a virus infection or a condition or symptom associated therewith, an IFN treatable disease or a condition in which the attenuated viruses can be used as a vector to induce an immune response to a particular antigen associated with the condition.

As used herein, and unless otherwise specified, the term “helix” means an object having a three-dimensional shape or “spiral” like that of a wire wound uniformly in a single layer around a cylinder or cone, as in a corkscrew or spiral staircase. It may or may not have a constant radius, that is, all the points on it are at the same distance from the axis. Alternative synonymous terms may comprise spiral corkscrew, curl, curlicue, twist, gyre, whorl, convolution, serpentine, etc. Geometrically a helix, as used herein can also mean a curve on a conical or cylindrical surface that would become a straight line if the surface were unrolled into a plane.

As used herein, and unless otherwise specified, the term “spiral” means a curve in a plane winding around a central point, or it could be a helix in space.

As used herein, and unless otherwise specified, the term “pitch”, “helix pitch”, and similar terms, refers to the diameter of the circle formed by the projection of a helix on a surface perpendicular to it. Further the “pitch: is the distance between any two points on the helix that are exactly 1 turn apart, measured parallel to the axis. The pitch of a helix is the height of one complete helix turn, measured parallel to the axis of the helix. A double helix consists of two (typically congruent) helices with the same axis, differing by a translation along the axis.

As used herein, and unless otherwise specified, the term “cylindrical” means having straight parallel sides and a circular or oval cross-section; in the shape or form of a cylinder; i.e.: “a cylindrical plastic container”.

As used herein, and unless otherwise specified, the term “multidirectional” means involving, or operating in several directions, such as moving around in a curved, non-linear or radial direction, such as around a column, a cylinder, a cone, a barrel shape, a parabolic, an ellipse, a hyperbola, etc., as non-limiting examples.

As used herein, and unless otherwise specified, the term “lysis buffer” refers to a buffer solution used to re-suspend and break open cells.

As used herein, and unless otherwise specified, the term “elution buffer” refers to a buffer solution used to remove a tagged protein of interest from a column.

As used herein, and unless otherwise specified, the term “pitch”, “thread pitch, or “channel pitch” means the distance between channels or threads expressed in millimeters (ISO standard—measured along the length of the column or cylinder). For example, a pitch of 1.5 means that the distance between one channel or thread and the next channel or thread is 1.5 mm. (In the Unified Thread Standard (UTS), which defines a standard thread form and series, a UTS thread is a number indicating the nominal (major) diameter of the thread, followed by the pitch measured in threads per inch. It has the same 60° profile as the ISO metric screw thread, but the characteristic dimensions of each UTS thread (outer diameter and pitch) were chosen as an inch fraction rather than a millimeter value).

As used herein, and unless otherwise specified, the term “radius of curvature” refers to the radius of a circle or helix that touches a curve at a given point and has the same tangent and curvature at that point.

The methods provided herein isolate one or more target(s) of interest from flowing matter using fluidic devices that detect, capture, adsorb, and/or remove the target(s) of interest (alternatively called “disease-causing material(s)” or “material(s) of interest”) from biological fluids and the subsequent diagnosis and treatment of disease based thereon. Devices are provided herein that are effective in and configured to isolate target(s) of interest from flowing matter by detecting, capturing, adsorbing, and/or removing target(s) of interest from biological fluids. The devices may further be configured to evaluate and/or identify the target(s) of interest and/or diagnose a disease based on the target(s) of interest isolated from the biological fluid. The devices may further be configured to provide information about the target(s) of interest to a system for evaluation and/or identification of the target(s) of interest. The target(s) of interest isolated by the device may be sufficiently preserved by the devices and methods herein that the disease-causing material may be transferred to and evaluated by a second system or device by traditional methods known to one of skill in the art.

The detection, capture, adsorption, and/or removal of the disease-causing material from the biological fluids itself may treat the disease in a subject and/or reduce or eliminate symptoms of the disease in the subject when the biological fluid (now having a reduced amount of disease-causing material therein, or now having the disease-causing material removed therefrom) is returned to the subject. As described herein, the device comprising the multidirectional channel is configurable for use to continuously filter a patient's blood in order to remove pathogens/endotoxins, thereby reducing the pathogen load and limiting the sepsis response. In some embodiments, the detection, capture, adsorption, and/or removal of the disease-causing material from the biological fluids provide practitioners with sufficient information to treat a disease in a subject or symptoms thereof, without need for further identification of the disease-causing material. In some embodiments, the detection, capture, adsorption, and/or removal of the disease-causing material from the biological fluids is followed by evaluation of the disease-causing material isolated by and eluted by the device. In some embodiments, the evaluation and/or analysis of the eluted materials of interest (or portions thereof or altered versions thereof) are conducted by the device or by other systems or devices which receive the eluted materials of interest, or a portion thereof, or an altered form thereof, or receive information about the eluted materials of interest. In some embodiments, the identification of the disease-causing material or the disease by the device or by another device receiving the disease-causing material, a portion thereof, an altered form of the disease-causing material, or information about the disease-causing material, is used to reduce or eliminate symptoms of the disease in the subject. In some embodiments, knowledge of a substance coated on the inner wall of a channel of a device combined with a determination that the substance captured, isolated, adsorbed or otherwise detected a material from the biological fluid passed through the channel is sufficient to diagnose a disease, determine a treatment for the subject, without further evaluation of the material isolated by the substance.

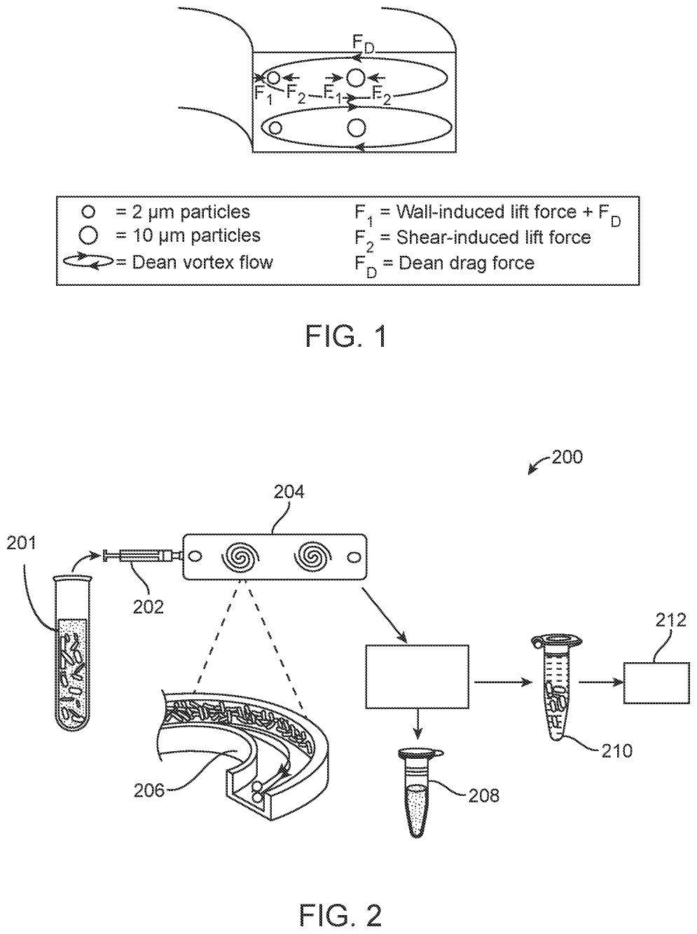

The presently described embodiments relate to a membrane-free pathogen adsorption fluidic cartridge technology that is capable of continuous flow. The working principle relies on three components: 1) polypeptide antibiotics bonded to the fluidic cartridge channel walls, 2) fluidic flow in curved or multidirectional channel structures, such as a spiral or helix, eliminating the need for membrane-based filters or external forces, and 3) the elution/lysis buffer (eluent or eluant) that allows for the elution of the materials of interest bound to the channel wall(s). FIG. 1 is a representation of particles flowing through a channel and associated force. Transverse force in a multidirectional channel, such as a spiral or helix, concentrates particulates according to the designed channel aspect ratio. Transverse Dean flows exert a Dean drag force (FD) on particles flowing in a spiral channel. The competition between wall lift force, shear lift force, and Dean drag force results in differential migration of particles to unique equilibrium positions dependent of particle size (Johnston, I. D. et al. Dean flow focusing and separation of small microspheres within a narrow size range. Microfluid. Nanofluidics 17, 509-518 (2014)). Concentrating particulates, such as bacteria, near the multidirectional channel sidewalls functionalized with polypeptide antibiotics promotes capture, adsorption, and removal of these particulates from fluid through interaction with the polypeptide antibiotics. The simplicity of this design (i.e. no moving parts, no membrane-based filter, etc.) allows for incorporation of this fluidic cartridge into other downstream processes. This fluidic cartridge can also serve as stand-alone blood filtration fluidic cartridge for the diagnosis and treatment of bacteremia, endotoxemia, sepsis, and other blood-borne diseases.

The described embodiments use a curved channel of a spiral or helical fluidic cartridge to introduce centrifugal force upon particulates (i.e. bacteria and endotoxin) flowing in a fluid (i.e. blood, saline, urine) to facilitate improved separation, capture, and removal of such particulates from the fluid. As these particulates flow through the channel, the particulates are forced toward the channel sidewalls, in a position offset from the center of the channel. The combination of centrifugal and fluidic forces in a helical- or spiral-shaped multidirectional channel allows for concentration of particulates near the polypeptide antibiotic functionalized sidewalls for capture and adsorption of bacteria and endotoxin.

In one example, as illustrated in the non-limiting FIG. 2, a method 200 is described, wherein; a fluidic cartridge 204, functionalized along the spiral channel walls 206 with polypeptide antibiotics designed to capture bacteria and endotoxin, is injected with a sample 202 containing bacteria 201. The subject bacteria are captured along the walls of its coated spiral channels by the coated polypeptide antibiotics due to the Dean drag force (FD) on particles flowing in the spiral channel, as described previously. The process sample 208 is then discarded. Following processing through the fluidic cartridge, an elution/lysis buffer (comprising deionized water DiH2O (50%) and ethanol/ethyl alcohol EtOH (50%) is then flown through the fluidic cartridge using a syringe and incubated at room temperature for about 1 hour. The eluate 210 is collected from the device outlet, tested 212 and analyzed. Afterwards the channel is washed with EtOH and air dried.

The polypeptide antibiotics described herein, including polymyxin (for Gram-negative bacteria) and vancomycin (for Gram-positive bacteria), are antibiotic substances having a strong affinity for the outer membrane of various strains of Gram-positive and Gram-negative bacteria, as well as an affinity for endotoxins. This allows for capture and adsorption of these biologics.

In another example, also as illustrated in the non-limiting FIG. 2, the method 200 is described, wherein; a sample 202 is caused to flow through a fluidic cartridge 204 comprising a multi-directional fluidic channel between an inlet and an outlet, functionalized along the spiral channel walls 206 with polypeptide antibiotics designed to capture bacteria and endotoxin; adsorbing one or more materials of interest on at least one wall of the multi-directional fluidic channel 206. The multidirectional fluidic cartridge channel is washed with a wash buffer (eluant) to separate the adsorbed materials of interest from a residual sample matter that is not of interest; discarding said wash buffer containing the residual sample matter that is not of interest. The process sample 208 is then discarded. The washed multi-directional fluidic channel 206 containing the adsorbed materials of interest is then contacted with a flowing eluant through the entire fluidic cartridge between the inlet and the outlet, collecting and concentrating the materials of interest in an eluate; and the collected amount of the eluate 210 containing the one or more concentrated materials of interest eluted from the sample is saved for evaluation, testing and/or analysis. In some embodiments, the sample 202 comprises an additive before flowing through the fluidic cartridge. In some embodiments the additive comprises one or more of: an EDTA; a K₂EDTA; a heparin compound; a fluoride; an oxalate; a sodium citrate; a sodium polyanethol sulfonate (SPS); an Acid Citrate Dextrose Solution; a distilled H2O; or a saline.

In some embodiments, the eluant is an elution buffer. In some embodiments, the eluant is a lysis buffer. In some embodiments, the eluate containing the concentrated materials of interest is purified by introducing the eluate into a spin column. In some embodiments, the spin column is placed in a centrifuge for centrifugation. During centrifugation, target sample materials are entrapped in the spin column matrix, while other unwanted materials pass through the spin column matrix into the collection vial. The collection vial is discarded. The targeted, isolated sample material entrapped in the spin column matrix is then released from the matrix and collected into a clean collection vial by washing the matrix with ethyl alcohol.

In some embodiments, the collected isolated materials of interest are analyzed. In some embodiments, the collected isolated materials of interest (or portions thereof or altered versions thereof) are analyzed by other systems or devices which receive the eluted materials of interest, or a portion thereof, or an altered form thereof, or receive information about the eluted materials of interest. In some embodiments, identification of the disease-causing material or the disease by the device or by another device receiving the disease-causing material, a portion thereof, an altered form of the disease-causing material, or information about the disease-causing material, is used to reduce or eliminate symptoms of the disease in the subject.

In some embodiments, the multi-directional fluidic channel is composed of at least one thermoplastic polymer base material that has at least one surface exposed functional group. In some embodiments, the thermoplastic polymer base material comprises: at least one exposed surface exposed wherein the thermoplastic polymer base material comprises at least one exposed surface selected from the functional group consisting of: a carbonyl group, a carboxyl group, an alcohol group, an amino group, a chloride group, a styrene group, an alpha-halogenated acyl group, a benzyl group, and an isocyanic acid groups, and a remaining thermoplastic polymer base material further comprises other polymers or copolymers comprising: a polyvinyl chloride, a polyvinyl acetate, a polyvinyl benzene, a polytetrafluoroethylene, a polyamide, an acrylamide, a polyurethane, a polyethylene, a polyethylene terephthalate, a polydimethylsiloxane, a polyacrylonitrile, a polycarbonate, an acetal, a polyethylene, a polypropylene, or a polymethyl methacrylate. In some embodiments, the thermoplastic polymer base material is polycarbonate.

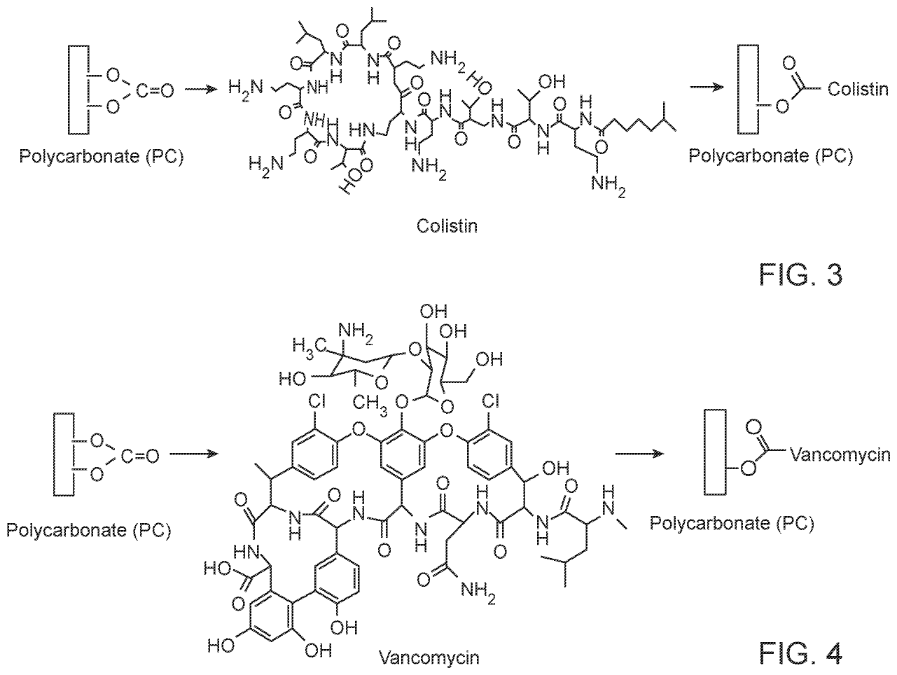

Among the materials listed, polycarbonate derivatives are suitable for the carrier due to the exposed carbonyl groups and biocompatibility. The exposed carbonyl groups on the polycarbonate surface undergo nucleophilic addition by the amine groups of the polypeptide antibiotic, such as colistin or vancomycin. This reaction results in scission of the polymer chain and the formation of a terminal carbamate. Addition of the polypeptide antibiotic containing one or more amine groups results in a polycarbonate surface decorated with the polypeptide antibiotic, which is available for subsequent capture of bacteria and/or endotoxin (FIG. 3 and FIG. 4).

In some embodiments, a substance is coated on the at least one wall of the multi-directional fluidic channel. FIG. 3 illustrates chemistry of functionalization of a polycarbonate surface with polymyxin E (i.e. colistin), in accordance with some embodiments. In some embodiments, a substance or a plurality of the substance is coated on at least one inside wall of the multi-directional fluidic channel. In some embodiments, the substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments, one or more substances or a plurality of the substances are coated on at least one wall of the multi-directional fluidic channel, wherein each substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments, the substance comprises an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent.

In some embodiments, the substance coating the channel wall comprises a fixed, covalently-bonded polypeptide antibiotic. In some embodiments, the fixed, covalently-bonded polypeptide antibiotic is polymyxin. In some embodiments, the amount of polymyxin fixed is about 0.5 mM to about 50 mM. In some embodiments, the amount of polymyxin fixed is at least about 0.5 mM. In some embodiments, the amount of polymyxin fixed is at most about 50 mM. In some embodiments, the amount of polymyxin fixed is about 0.5 mM to about 1 mM, about 0.5 mM to about 5 mM, about 0.5 mM to about 10 mM, about 0.5 mM to about 20 mM, about 0.5 mM to about 30 mM, about 0.5 mM to about 40 mM, about 0.5 mM to about 50 mM, about 1 mM to about 5 mM, about 1 mM to about 10 mM, about 1 mM to about 20 mM, about 1 mM to about 30 mM, about 1 mM to about 40 mM, about 1 mM to about 50 mM, about 5 mM to about 10 mM, about 5 mM to about 20 mM, about 5 mM to about 30 mM, about 5 mM to about 40 mM, about 5 mM to about 50 mM, about 10 mM to about 20 mM, about 10 mM to about 30 mM, about 10 mM to about 40 mM, about 10 mM to about 50 mM, about 20 mM to about 30 mM, about 20 mM to about 40 mM, about 20 mM to about 50 mM, about 30 mM to about 40 mM, about 30 mM to about 50 mM, or about 40 mM to about 50 mM. The term “about” when used in reference to the amount of polymyxin, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the fixed, covalently-bonded polypeptide antibiotic is vancomycin. FIG. 4 illustrates chemistry of functionalization of polycarbonate surface with vancomycin, in accordance with some embodiments. In some embodiments, the amount of vancomycin fixed is about 0.5 mM to about 50 mM. In some embodiments, the amount of vancomycin fixed is at least about 0.5 mM. In some embodiments, the amount of vancomycin fixed is at most about 50 mM. In some embodiments, the amount of vancomycin fixed is about 0.5 mM to about 1 mM, about 0.5 mM to about 5 mM, about 0.5 mM to about 10 mM, about 0.5 mM to about 20 mM, about 0.5 mM to about 30 mM, about 0.5 mM to about 40 mM, about 0.5 mM to about 50 mM, about 1 mM to about 5 mM, about 1 mM to about 10 mM, about 1 mM to about 20 mM, about 1 mM to about 30 mM, about 1 mM to about 40 mM, about 1 mM to about 50 mM, about 5 mM to about 10 mM, about 5 mM to about 20 mM, about 5 mM to about 30 mM, about 5 mM to about 40 mM, about 5 mM to about 50 mM, about 10 mM to about 20 mM, about 10 mM to about 30 mM, about 10 mM to about 40 mM, about 10 mM to about 50 mM, about 20 mM to about 30 mM, about 20 mM to about 40 mM, about 20 mM to about 50 mM, about 30 mM to about 40 mM, about 30 mM to about 50 mM, or about 40 mM to about 50 mM. The term “about” when used in reference to the amount of vancomycin, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

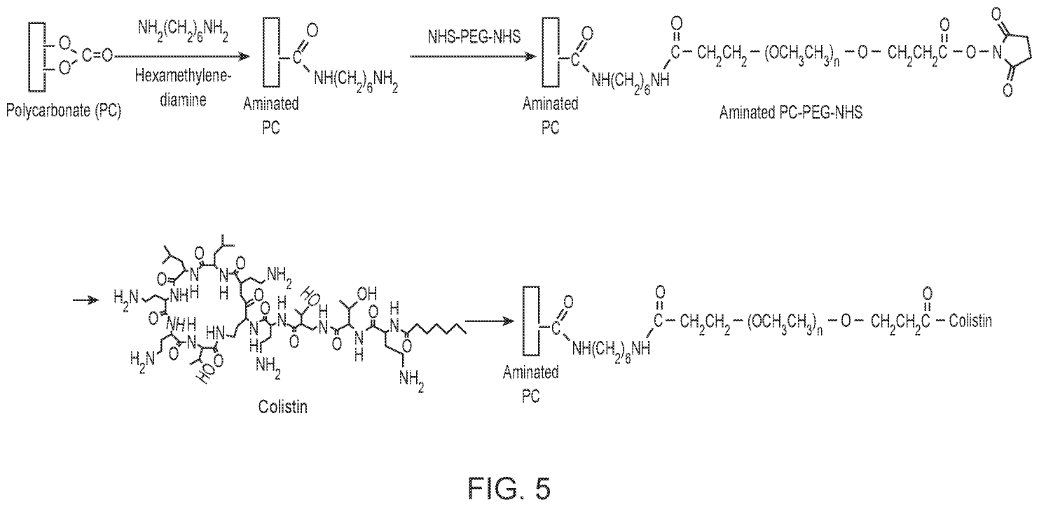

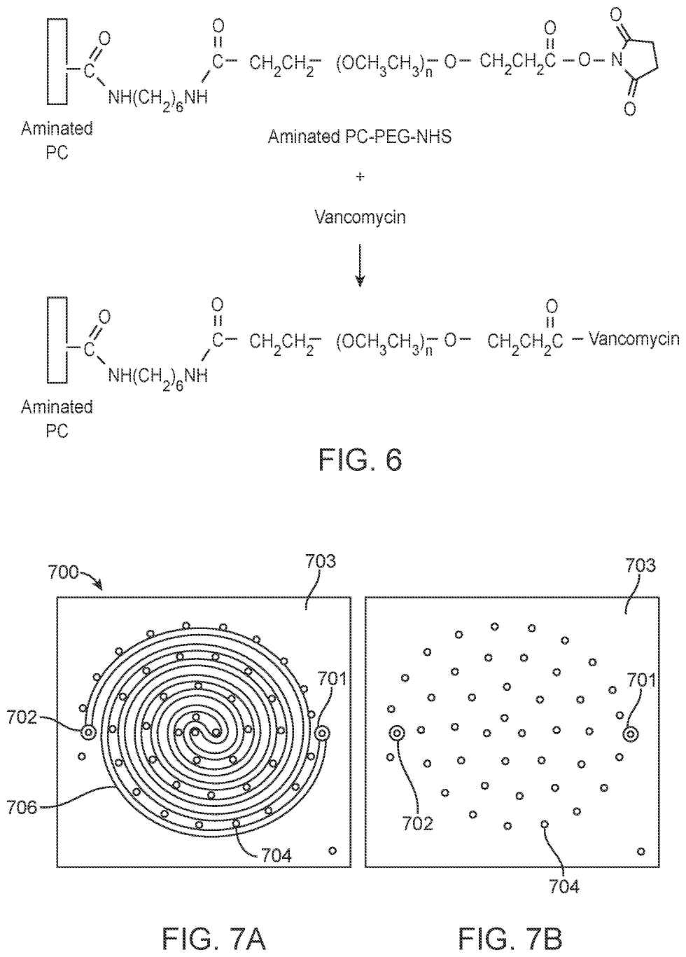

FIG. 5 illustrates chemistry of functionalization of polycarbonate surface with a crosslinking agent and polymyxin E (i.e. colistin), in accordance with some embodiments. FIG. 6 illustrates chemistry of functionalization of polycarbonate surface with a crosslinking agent and, in accordance with some embodiments. In some embodiments, the crosslinking agent to be fixed to the base material is polyethylene glycol or a derivative substance in which the amount fixed is about 1 mM to about 50 mM. In some embodiments, the crosslinking agent to be fixed to the base material is polyethylene glycol or a derivative substance in which the amount fixed is at least about 1 mM. In some embodiments, the crosslinking agent to be fixed to the base material is polyethylene glycol or a derivative substance in which the amount fixed is at most about 50 mM. In some embodiments, the crosslinking agent to be fixed to the base material is polyethylene glycol or a derivative substance in which the amount fixed is about 1 mM to about 5 mM, about 1 mM to about 10 mM, about 1 mM to about 20 mM, about 1 mM to about 30 mM, about 1 mM to about 40 mM, about 1 mM to about 50 mM, about 5 mM to about 10 mM, about 5 mM to about 20 mM, about 5 mM to about 30 mM, about 5 mM to about 40 mM, about 5 mM to about 50 mM, about 10 mM to about 20 mM, about 10 mM to about 30 mM, about 10 mM to about 40 mM, about 10 mM to about 50 mM, about 20 mM to about 30 mM, about 20 mM to about 40 mM, about 20 mM to about 50 mM, about 30 mM to about 40 mM, about 30 mM to about 50 mM, or about 40 mM to about 50 mM. The term about, when used in reference to the amount of crosslinking agent, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

As illustrated in FIG. 3 and FIG. 4, the multidirectional channel is made of a base material that is typically a thermoplastic polymer, such as polycarbonate 300, 400. The thermoplastic polymer preferably has surface exposed functional groups capable of fixing polymyxin 401, vancomycin 301, and other relative molecules. Examples of the exposed functional groups characteristic of the base material include carbonyl groups, carboxyl groups, alcohol groups, amino groups, chloride groups, styrene groups, alpha-halogenated acyl group, benzyl groups, isocyanic acid groups, and other polymers or copolymers such as vinylchloride, vinylacetate, acrylamide, polyethylene, polyethylene terephthalate acrylic acid, acrylonitrile, maleic anhydride, methylmethacrylate, etc. Among the materials listed, polycarbonate derivatives are most suitable for the carrier due to the exposed carbonyl groups and biocompatibility. The exposed carbonyl groups on the polycarbonate surface undergo nucleophilic addition by the amine groups of the polypeptide antibiotic, such as colistin or vancomycin. This reaction results in scission of the polymer chain and the formation of a terminal carbamate. Addition of the polypeptide antibiotic containing one or more amine groups results in a polycarbonate surface decorated with the polypeptide antibiotic 402, 302, which is available for subsequent capture of bacteria and/or endotoxin (as illustrated in the non-limiting FIG. 3 and FIG. 4).

In one example (FIG. 3), Colistin sulfate salt 301 (0.28 g) was added to 16 mLs of an ethanol/water mixture (50% DiH2O/50% EtOH). The colistin solution was added to the polycarbonate-based channel 300 and incubated within the channel at room temperature for 1 hour. Afterwards, the channel was washed with EtOH and air dried.

In another example (FIG. 4), Vancomycin 401 (0.22 g) was added to 13 mLs of an ethanol/water mixture (50% DiH2O/50% EtOH (v/v)). The vancomycin solution was added to the polycarbonate-based channel 401 and incubated within the channel at room temperature for 1 hour. Afterwards, the channel was washed with EtOH and air dried.

The amount of polypeptide antibiotic substance to be fixed to the base material is, preferably, more than 0.5 mM of substance and more preferably, 1.0-50.0 mM substance. If the amount is less than 0.5 mM of substance, detoxifying will potentially become ineffective.

In some embodiments, the fluidic cartridge is disposable.

The multidirectional channel preferably has a surface area of 0.001 to 100 m², more preferably 0.001 to 10 m², as well as exposed functional groups being capable of fixing polymyxin and vancomycin. A surface area too large increases the pressure drop too much, and a surface area that is too small makes the isolation capacity insufficient. The pressure drop is most preferably less than 10 psi, and most preferably less than 3 psi.

In one embodiment, the spiral-shaped multidirectional channel preferably has two, 1 to 10-loop spiral channels joined at an S-junction to form a double spiral channel with one inlet and one outlet for bacterial/endotoxin removal. More preferably, in another embodiment, the spiral-shaped multidirectional channel has two, 3 to 6-loop spiral channels joined at an S-junction to form a double spiral channel with one inlet and one outlet for bacterial/endotoxin isolation.

In some embodiments of the fluidic cartridge, the multidirectional channel has an internal width of about 0.001 mm to about 1,000 mm. In some embodiments of the fluidic cartridge, the multidirectional channel has an internal width of at least about 0.001 mm. In some embodiments of the fluidic cartridge, the multidirectional channel has an internal width of at most about 1,000 mm. In some embodiments of the fluidic cartridge, the multidirectional channel has an internal width of about 0.001 mm to about 0.005 mm, about 0.001 mm to about 0.1 mm, about 0.001 mm to about 0.05 mm, about 0.001 mm to about 1 mm, about 0.001 mm to about 10 mm, about 0.001 mm to about 50 mm, about 0.001 mm to about 100 mm, about 0.001 mm to about 500 mm, about 0.001 mm to about 1,000 mm, about 0.005 mm to about 0.1 mm, about 0.005 mm to about 0.5 mm, about 0.005 mm to about 1 mm, about 0.005 mm to about 10 mm, about 0.005 mm to about 50 mm, about 0.005 mm to about 100 mm, about 0.005 mm to about 500 mm, about 0.005 mm to about 1,000 mm, about 0.01 mm to about 0.5 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.05 mm to about 1 mm, about 0.05 mm to about 10 mm, about 0.05 mm to about 50 mm, about 0.05 mm to about 100 mm, about 0.05 mm to about 500 mm, about 0.05 mm to about 1,000 mm, about 0.1 mm to about 10 mm, about 0.1 mm to about 50 mm, about 0.1 mm to about 100 mm, about 0.1 mm to about 500 mm, about 0.1 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the internal width of the multi-directional channel, in some embodiments means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the width of the multi-directional channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm or 0.01 mm of a given value or range.

In some embodiments of the fluidic cartridge, the multidirectional channel has an internal height of about 0.001 mm to about 100 mm. In some embodiments of the fluidic cartridge, the multidirectional channel has an internal height of at least about 0.001 mm. In some embodiments of the fluidic cartridge, the multidirectional channel has an internal height of at most about 100 mm. In some embodiments of the fluidic cartridge, the multidirectional channel has an internal height of about 0.001 mm to about 0.005 mm, about 0.001 mm to about 0.01 mm, about 0.001 mm to about 0.05 mm, about 0.001 mm to about 0.1 mm, about 0.001 mm to about 0.5 mm, about 0.001 mm to about 1 mm, about 0.001 mm to about 10 mm, about 0.001 mm to about 50 mm, about 0.001 mm to about 100 mm, about 0.005 mm to about 0.01 mm, about 0.005 mm to about 0.05 mm, about 0.005 mm to about 0.1 mm, about 0.005 mm to about 0.5 mm, about 0.005 mm to about 1 mm, about 0.005 mm to about 10 mm, about 0.005 mm to about 50 mm, about 0.005 mm to about 100 mm, about 0.01 mm to about 0.05 mm, about 0.01 mm to about 0.1 mm, about 0.01 mm to about 0.5 mm, about 0.01 mm to about 1 mm, about 0.01 mm to about 10 mm, about 0.01 mm to about 50 mm, about 0.01 mm to about 100 mm, about 0.05 mm to about 0.1 mm, about 0.05 mm to about 0.5 mm, about 0.05 mm to about 1 mm, about 0.05 mm to about 10 mm, about 0.05 mm to about 50 mm, about 0.05 mm to about 100 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.5 mm to about 1 mm, about 0.5 mm to about 10 mm, about 0.5 mm to about 50 mm, about 0.5 mm to about 100 mm, about 1 mm to about 10 mm, about 1 mm to about 50 mm, about 1 mm to about 100 mm, about 10 mm to about 50 mm, about 10 mm to about 100 mm, or about 50 mm to about 100 mm. The term “about”, when used in reference to the height of the multidirectional channel, in some embodiments, means 0.001 mm, 0.005 mm, 0.01 mm, 0.05 mm, 0.1 mm, 0.5 mm, 1 mm, 10 mm, or 50 mm. The term “about”, when used in reference to the height of the multi-directional channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the height of the multi-directional channel, means within 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm or 0.01 mm of a given value or range.

In some embodiments of the fluidic cartridge, the multidirectional channel has a length of about 0.01 mm to about 10,000 mm between the inlet and the outlet. In some embodiments of the fluidic cartridge, the multidirectional channel has a length of at least about 0.01 mm between the inlet and the outlet. In some embodiments of the fluidic cartridge, the multidirectional channel has a length of at most about 10,000 mm between the inlet and the outlet. In some embodiments of the fluidic cartridge, the multidirectional channel has a length between the inlet and the outlet of about 0.001 mm to about 0.05 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.01 mm to about 5,000 mm, about 0.01 mm to about 10,000 mm, about 0.05 mm to about 1 mm, about 0.05 mm to about 10 mm, about 0.05 mm to about 50 mm, about 0.50 mm to about 100 mm, about 0.05 mm to about 500 mm, about 0.05 mm to about 1,000 mm, about 0.05 mm to about 5,000 mm, about 0.05 mm to about 10,000 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.1 mm to about 500 mm, about 0.1 mm to about 1,000 mm, about 0.1 mm to about 5,000 mm, about 0.1 mm to about 10,000 mm, about 0.5 mm to about 1 mm, about 0.5 mm to about 10 mm, about 0.5 mm to about 50 mm, about 0.5 mm to about 100 mm, about 0.5 mm to about 500 mm, about 0.5 mm to about 1,000 mm, about 0.5 mm to about 5,000 mm, about 0.5 mm to about 10,000 mm, about 1 mm to about 10 mm, about 1 mm to about 50 mm, about 1 mm to about 100 mm, about 1 mm to about 500 mm, about 1 mm to about 1,000 mm, about 1 mm to about 5,000 mm, about 1 mm to about 10,000 mm, about 10 mm to about 50 mm, about 10 mm to about 100 mm, about 10 mm to about 500 mm, about 10 mm to about 1,000 mm, about 10 mm to about 5,000 mm, about 10 mm to about 10,000 mm, about 50 mm to about 100 mm, about 50 mm to about 500 mm, about 50 mm to about 1,000 mm, about 50 mm to about 5,000 mm, about 50 mm to about 10,000 mm, about 100 mm to about 500 mm, about 100 mm to about 1,000 mm, about 100 mm to about 5,000 mm, about 100 mm to about 10,000 mm, about 500 mm to about 1,000 mm, about 500 mm to about 5,000 mm, about 500 mm to about 10,000 mm, about 1,000 mm to about 5,000 mm, about 1,000 mm to about 10,000 mm, or about 5,000 mm to about 10,000 mm. The term “about”, when used in reference to the length of the multi-directional channel between the inlet and the outlet, in some embodiments means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the length of the multi-directional channel, means within 5,000 mm, 1,000 mm, 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments of the fluidic cartridge, the multidirectional channel has a length of about 0.1 mm to about 3,000.0 mm between the inlet and the outlet. In some embodiments of the fluidic cartridge, the multidirectional channel has a length of at least about 0.01 mm between the inlet and the outlet. In some embodiments of the fluidic cartridge, the multidirectional channel has a length of at most about 3,000 mm between the inlet and the outlet. In some embodiments of the fluidic cartridge, the multidirectional channel has a length between the inlet and the outlet of about 0.001 mm to about 0.05 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.01 mm to about 2,000 mm, about 0.01 mm to about 3,000 mm, about 0.05 mm to about 1 mm, about 0.05 mm to about 10 mm, about 0.05 mm to about 50 mm, about 0.50 mm to about 100 mm, about 0.05 mm to about 500 mm, about 0.05 mm to about 1,000 mm, about 0.05 mm to about 2,000 mm, about 0.05 mm to about 3,000 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.1 mm to about 500 mm, about 0.1 mm to about 1,000 mm, about 0.1 mm to about 2,000 mm, about 0.1 mm to about 3,000 mm, about 0.5 mm to about 1 mm, about 0.5 mm to about 10 mm, about 0.5 mm to about 50 mm, about 0.5 mm to about 100 mm, about 0.5 mm to about 500 mm, about 0.5 mm to about 1,000 mm, about 0.5 mm to about 2,000 mm, about 0.5 mm to about 3,000 mm, about 1 mm to about 10 mm, about 1 mm to about 50 mm, about 1 mm to about 100 mm, about 1 mm to about 500 mm, about 1 mm to about 1,000 mm, about 1 mm to about 2,000 mm, about 1 mm to about 3,000 mm, about 10 mm to about 50 mm, about 10 mm to about 100 mm, about 10 mm to about 500 mm, about 10 mm to about 1,000 mm, about 10 mm to about 2,000 mm, about 10 mm to about 3,000 mm, about 50 mm to about 100 mm, about 50 mm to about 500 mm, about 50 mm to about 1,000 mm, about 50 mm to about 2,000 mm, about 50 mm to about 3,000 mm, about 100 mm to about 500 mm, about 100 mm to about 1,000 mm, about 100 mm to about 2,000 mm, about 100 mm to about 3,000 mm, about 500 mm to about 1,000 mm, about 500 mm to about 2,000 mm, about 500 mm to about 3,000 mm, about 1,000 mm to about 2,000 mm, about 1,000 mm to about 3,000 mm, or about 2,000 mm to about 3,000 mm. The term “about”, when used in reference to the length of the multi-directional channel between the inlet and the outlet, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the length of the multi-directional channel, means within 1,500 mm, 1,000 mm, 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments, said multi-directional channel is spiral shaped. In some embodiments of the fluidic cartridge, the spiral shaped channel has a radius of curvature ranging between about 0.01 mm to about 1,000 mm. In some embodiments of the fluidic cartridge, the spiral shaped channel has an inner-most radius of curvature at least about 0.01 mm. In some embodiments of the fluidic cartridge, the spiral shaped channel has an outer-most radius of curvature at most about 1,000 mm. In some embodiments of the fluidic cartridge, the spiral shaped channel has a radius of curvature between about 0.01 mm to about 10 mm, about 0.01 mm to about 50 mm, about 0.01 mm to about 100 mm, about 0.01 mm to about 500 mm, about 0.01 mm to about 1,000 mm, about 0.1 mm to about 10 mm, about 0.1 mm to about 50 mm, about 0.1 mm to about 100 mm, about 0.1 mm to about 500 mm, about 0.1 mm to about 1,000 mm, about 1 mm to about 10 mm, about 1 mm to about 50 mm, about 1 mm to about 100 mm, about 1 mm to about 500 mm, about 1 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the radius of curvature of the spiral shaped channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the outer-most radius of curvature of the spiral shaped channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments, the spiral shaped channel has a radius of curvature ranging between about 5 mm to about 100.0 mm. In some embodiments of the fluidic cartridge, the spiral shaped channel has an inner-most radius of curvature of at least about 5 mm. In some embodiments of the fluidic cartridge, the spiral shaped channel has an outer-most radius of curvature of at most about 100 mm. In some embodiments of the fluidic cartridge, the spiral shaped channel has a radius of curvature between about 5 mm to about 10 mm, about 5 mm to about 25 mm, about 5 mm to about 50 mm, about 5 mm to about 75 mm, about 5 mm to about 100 mm, about 10 mm to about 25 mm, about 10 mm to about 50 mm, about 10 mm to about 75 mm, about 10 mm to about 100 mm, about 25 mm to about 50 mm, about 25 mm to about 75 mm, about 25 mm to about 100 mm, about 50 mm to about 75 mm, about 50 mm to about 100 mm, or about 75 mm to about 100 mm. The term “about”, when used in reference to the radius of curvature of the spiral shaped channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the outer-most radius of curvature of the spiral shaped channel, means within 5.0 mm, 2.5 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments, the spiral shaped multi-directional channel has a distance between channel edges in the spiral of about 0.01 to about 1,000 mm. In some embodiments of the fluidic cartridge, the spiral shaped multidirectional channel has a distance between the channel edges in the spiral of at least about 0.01 mm. In some embodiments of the fluidic cartridge, the spiral shaped multidirectional channel has a distance between the channel edges in the spiral of at most about 1,000 mm. In some embodiments of the fluidic cartridge, the spiral shaped multidirectional channel has a distance between the channels in the spiral about 0.01 mm to about 0.05 mm, about 0.01 mm to about 0.1 mm, about 0.01 mm to about 0.5 mm, about 0.01 mm to about 1 mm, about 0.01 mm to about 10 mm, about 0.01 mm to about 50 mm, about 0.01 mm to about 100 mm, about 0.01 mm to about 500 mm, about 0.01 mm to about 1,000 mm, about 0.05 mm to about 0.1 mm, about 0.05 mm to about 0.5 mm, about 0.05 mm to about 1 mm, about 0.05 mm to about 10 mm, about 0.05 mm to about 50 mm, about 0.05 mm to about 100 mm, about 0.05 mm to about 500 mm, about 0.05 mm to about 1,000 mm, about 0.1 mm to about 0.5 mm, about 0.1 mm to about 1 mm, about 0.1 mm to about 10 mm, about 0.1 mm to about 50 mm, about 0.1 mm to about 100 mm, about 0.1 mm to about 500 mm, about 0.1 mm to about 1,000 mm, about 0.5 mm to about 1 mm, about 0.5 mm to about 10 mm, about 0.5 mm to about 50 mm, about 0.5 mm to about 100 mm, about 0.5 mm to about 500 mm, about 0.5 mm to about 1,000 mm, about 1 mm to about 10 mm, about 1 mm to about 50 mm, about 1 mm to about 100 mm, about 1 mm to about 500 mm, about 1 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the distance between the channel edges in the spiral, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the distance between the channels in the spiral, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 2.5 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In a further embodiment, the helical-shaped multidirectional channel is preferably fabricated around a cylindrical chamber. The vertically designed fluidic cartridge offers a constant radius of curvature and compact size and shape. The helical-shaped multidirectional channel preferably has 3 to 30 loops fabricated around a cylindrical chamber with one inlet and one outlet for bacterial/endotoxin removal. More preferably, the helical-shaped multidirectional channel is composed of 3 to 20-loops fabricated around a cylindrical chamber with one inlet and one outlet for bacterial/endotoxin removal.

In some embodiments, said multi-directional channel is helically shaped. In some embodiments, said multi-directional channel is fabricated around a cylindrical chamber. In some embodiments, said multi-directional channel is helically shaped and fabricated around a cylindrical chamber. In some embodiments of the fluidic cartridge, the helical shaped multidirectional channel has a radius of curvature ranging between about 1 mm to about 1,000 mm. In some embodiments of the fluidic cartridge, the helical shaped multidirectional channel has a radius of curvature of at least about 1 mm. In some embodiments of the fluidic cartridge, the helical shaped multidirectional channel has a radius of curvature of at most about 1,000 mm. In some embodiments of the fluidic cartridge, the helical shaped multidirectional channel has a radius of curvature ranging between about 1 mm to about 5 mm, about 1 mm to about 10 mm, about 1 mm to about 50 mm, about 1 mm to about 100 mm, about 1 mm to about 500 mm, about 1 mm to about 1,000 mm, about 5 mm to about 10 mm, about 5 mm to about 50 mm, about 5 mm to about 100 mm, about 5 mm to about 500 mm, about 5 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the radius of curvature of the helical shaped multidirectional channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the radius of curvature of the helical shaped multidirectional channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 2.5 mm, 1.0 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, 0.009 mm, 0.008 mm, 0.007 mm, 0.006 mm, 0.005 mm, 0.004 mm, 0.003 mm, 0.002 mm, or 0.001 mm of a given value or range.

In some embodiments of the fluidic cartridge, the helical shaped multidirectional channel has a pitch between about 1 mm to about 1,000 mm. In some embodiments of the fluidic cartridge, the helical shaped multidirectional channel has a pitch of at least about 1 mm. In some embodiments of the fluidic cartridge, the helical shaped multidirectional channel has a pitch of at most about 1,000 mm. In some embodiments of the fluidic cartridge, the helical shaped multidirectional channel has a pitch between about 1 mm to about 5 mm, about 1 mm to about 10 mm, about 1 mm to about 50 mm, about 1 mm to about 100 mm, about 1 mm to about 500 mm, about 1 mm to about 1,000 mm, about 5 mm to about 10 mm, about 5 mm to about 50 mm, about 5 mm to about 100 mm, about 5 mm to about 500 mm, about 5 mm to about 1,000 mm, about 10 mm to about 50 mm, about 10 mm to about 100 mm, about 10 mm to about 500 mm, about 10 mm to about 1,000 mm, about 50 mm to about 100 mm, about 50 mm to about 500 mm, about 50 mm to about 1,000 mm, about 100 mm to about 500 mm, about 100 mm to about 1,000 mm, or about 500 mm to about 1,000 mm. The term “about”, when used in reference to the pitch of the helical shaped multidirectional channel, in some embodiments, means within 50%, 40%, 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about”, when used in reference to the pitch of the helical shaped multidirectional channel, means within 500.0 mm, 400.0 mm, 300.0 mm, 200.0 mm, 100.0 mm, 90.0 mm, 80.0 mm, 70.0 mm, 60.0 mm, 50.0 mm, 40.0 mm, 30.0 mm, 20.0 mm, 10.0 mm, 5.0 mm, 2.5 mm, 1.0 mm, 0.9 mm, 0.8 mm, 0.7 mm, 0.6 mm, 0.5 mm, 0.4 mm, 0.3 mm, 0.2 mm or 0.1 mm of a given value or range.

In some embodiments, helical-shaped multidirectional channel comprises an inner sleeve on a cylinder. In some embodiments the helical-shaped multidirectional channel comprises an outer sleeve on a cylinder. In some embodiments, the multi-directional channel is enclosed. In some embodiments, the multi-directional channel is enclosed between the at least one inlet and the at least one outlet. In some embodiments, the multi-directional channel is enclosed between the at least one inlet and the at least one outlet on the inside of a cylinder. In some embodiments, the multi-directional channel is enclosed between the at least one inlet and the at least one outlet on the outside of a cylinder. In some embodiments, the multi-directional channel is a multidirectional helix enclosed between the at least one inlet and the at least one outlet on the inside and the outside of a cylinder.

In some embodiments, the multi-directional channel is enclosed using at least one of: bolts, an adhesive, a binding material, a resin, an inner sleeve and an outer sleeve; utilizing at least one of: a base plate, a cover glass, a curing (method), a thermal expansion (process), ultrasonic welding, vibration welding, high frequency welding—(aka: radio frequency welding, and dielectric welding), heated tool or plate welding, solvent bonding, laser welding, spin welding, infrared welding, and adhesive bonding.

In some embodiments, the fluidic cartridge comprising the multi-directional channel is fabricated using at least one method selected from the group consisting of: 3-D printing, stereolithography, photolithography, injection molding, blow molding, casting, ultrasonic welding, vibration welding, high frequency welding—(aka: radio frequency welding, and dielectric welding), heated tool or plate welding, solvent bonding, laser welding, spin welding, infrared welding, adhesive bonding, machining, turning, drilling, boring, reaming, electric discharge machining (EDM) and milling.

The helical-shaped multidirectional channel preferably has a radius of curvature of about 1.0 mm to about 1,000 mm, more preferably about 10.0 mm to about 500 mm, and still more preferably about 5.0 mm to about 100 mm.

The pitch of the helical channel preferably about 1.0 mm to about 1,000 mm, more preferably about 10.0 mm to about 500 mm, and still more preferably about 10.0 mm to about 100 mm.

The spiral-shaped multidirectional channel preferably has an outer-most radius of curvature of about 0.1 mm to about 1,000 mm, more preferably about 0.1 mm to about 100 mm, and still more preferably about 1.0 mm to about 100 mm.

The spiral-shaped multidirectional channel preferably has a distance between channels of about 0.01 mm to about 1,000 mm, more preferably about 0.1 mm to about 100 mm, and still more preferably 0.1 about mm to about 10.0 mm.

The helical- and spiral-shaped multidirectional channel(s) preferably have a channel width of about 0.01 mm to about 1,000 mm, more preferably about 0.01 mm to about 100 mm, and still more preferably about 0.01 mm to about 10.0 mm.

The helical- and spiral-shaped multidirectional channel(s) preferably have a channel height of about 0.001 mm to about 100 mm, more preferably about 0.001 mm to about 20.0 mm, and still more preferably about 0.001 mm to about 10.0 mm.

The helical- and spiral-shaped multidirectional channel(s) preferably have a channel length of about 0.01 mm to about 10,000 mm, more preferably about 0.01 mm to about 5,000 mm, and still more preferably about 0.01 mm to 3,000 mm.

FIG. 7A and FIG. 7B illustrate a non-limiting embodiment of a spiral-shaped multidirectional channel 700, in accordance with some embodiments. As shown therein, a series of spiral pivot points 704 are defined, either in a plane or a helix surrounding a cylinder, that determine the path of the representative fluidic cartridge channel 706. The representative fluidic channel 706 may be within or on a surface of a substrate or base 703. The inlet 701 and the outlet 702 of the channel are also illustrated. In some embodiments, the spiral-shaped multidirectional channel has two, 3 to 6-loop spiral channels joined at an S-junction to form a double spiral channel. In some embodiments, the helical-shaped multidirectional channel comprises less than 3 loops with one inlet and one outlet for bacterial/endotoxin removal. In some embodiments, the helical-shaped multidirectional channel comprises more than 20 loops with one inlet and one outlet for bacterial/endotoxin removal. Ideally, the helical-shaped multidirectional channel is configured to provide maximum exposure in both time and surface area to enable it to capture, adsorb, and/or remove disease-causing material from biological fluids. Alternatively, the ultimate length and/or number of loops must be monitored to assure that an excessive pressure drop does not occur when the device is used in an in-line procedure with a patient.

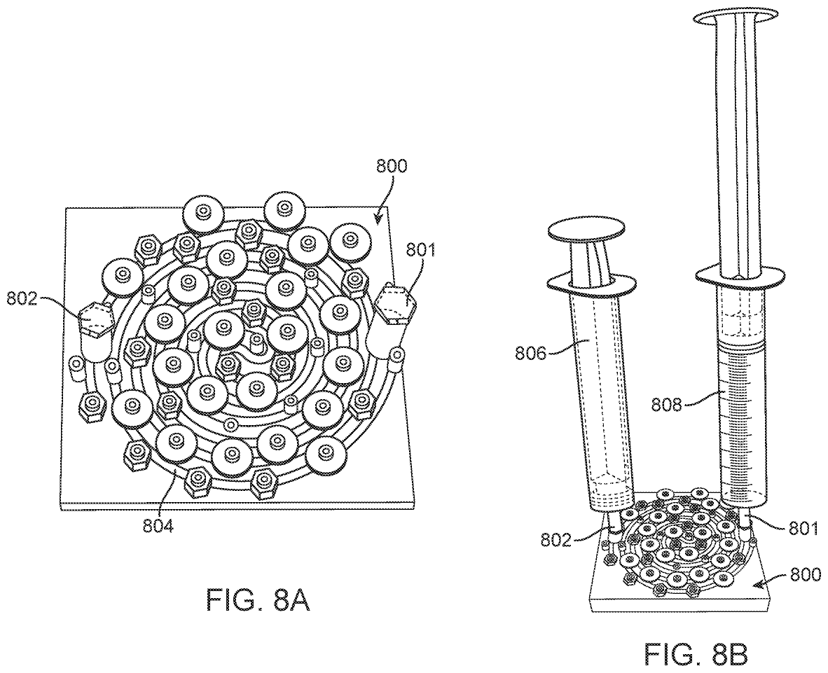

FIG. 8A and FIG. 8B illustrate a non-limiting example of a fluidic cartridge 800. The fluidic cartridge 800 is manufacturable through the employment of any number of technologies. For example, among the more common methods of manufacture are 3-D printing, stereolithography, photolithography, injection molding, blow molding, casting, ultrasonic welding, high frequency welding, heated tool or plate welding, solvent bonding, laser welding, spin welding, infrared welding, vibration welding, adhesive bonding, and machining. In some embodiments, the preferred machining methods are turning, drilling, boring, reaming, electric discharge machining (EDM) and/or milling. Material are commonly machined to create an enclosure of desired dimensions, such as by machining channel(s) as a whole or by machining halves that are then sealed and enclosed using bolts, adhesive, thermal expansion, or another means.

As shown in FIG. 8A, a multidirectional channel 804 may have a channel Length=837 mm; a Width=2400 μm; a Height=500 μm. The cartridge may be fabricated using milling techniques and sealed by bolting two polycarbonate pieces together. For the channel(s) 804, luer lock syringes 806, 808 are configurable to be attached to the inlet(s) 801 and outlet(s) 802 by the use of fittings, caps, or luer lock connectors. FIG. 8B illustrates a spiral-shaped multidirectional channel connected to the two syringes 806, 808 using luer lock fittings. The machining may be performed on various biocompatible materials, including various grades of polycarbonate. The channel of the fluidic cartridge is then be coated with a substance, such as polymyxin or vancomycin, using previously described methods. The coating should coat at least a portion of an inside wall of the multidirectional channel(s) through conventional means.

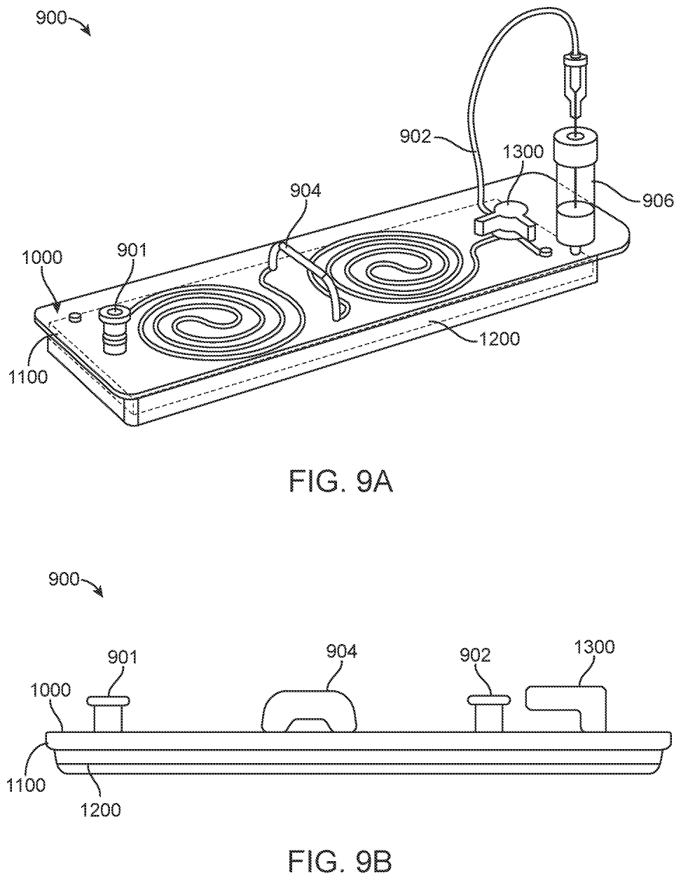

FIGS. 9A, 9B, 10-10C, 11A-11D, 12A-12C, and 13A-13C illustrate a non-limiting example of a device 900 comprising a multidirectional channel. FIG. 9A illustrates an isometric view of a device 900, in accordance with some embodiments. FIG. 9B illustrates a side view of a device 900, in accordance with some embodiments.

As shown in FIG. 9A and FIG. 9B, the device 900 may comprise inlet 901 and outlet 902. The device may comprise top plate 1000, bottom plate 1100, and reservoir 1200. The flow of a sample from the multidirectional channel to reservoir 1200 and outlet 902 may be controlled by valve 1300. In some embodiments, the spiral-shaped multidirectional channel has two, 1 to 6-loop spiral channels joined at an S-junction to form a double spiral channel. As shown, the device may comprise two spiral channels joined by junction 904. Junction 904 may be removable. In some cases, a first multidirectional channel may comprise colistin and a second may comprise vancomycin. In some cases, a first multidirectional channel may comprise vancomycin and a second may comprise colistin. The colistin and vancomycin may be added as described herein, for example, with respect to FIG. 3 and FIG. 4. The sample may flow from outlet 902 to collection vial 906.

As shown in FIG. 9A and FIG. 9B, a system 900 comprising a multidirectional channel may have a channel length of 333 mm per spiral, a channel width of 2 mm, a channel height of 0.5 mm. The width and height of a channel may be about 4 to 1. The channel dimensions of a device 900 may be sufficiently large such that pressure from a standard luer lock syringe may be used press a sample through the multidimensional channel. The outer most radius of curvature may be 22 mm.

The device 900 may be manufactured through any number of technologies. For example, among the more common methods of manufacture are 3-D printing, stereolithography, photolithography, injection molding, blow molding, casting, ultrasonic welding, high frequency welding, heated tool or plate welding, solvent bonding, laser welding, spin welding, infrared welding, vibration welding, adhesive bonding, and machining. In some embodiments, the preferred machining methods are turning, drilling, boring, reaming, electric discharge machining (EDM) and/or milling. Material are commonly machined to create an enclosure of desired dimensions, such as by machining channel(s) as a whole or by machining halves that are then sealed and enclosed using bolts, adhesive, thermal expansion, or another means. In an example, the device 900 may be fabricated by milling the top plate 1000, bottom plate 1100, and reservoir 1200 and gluing or bolting the respective pieces together. Top plate 1000, bottom plate 1100, and reservoir 1200 of the device 900 may comprise polycarbonate. In some embodiments, the multidirectional channel formed by top plate 1000 and bottom plate 1100 may comprise any material as described herein.

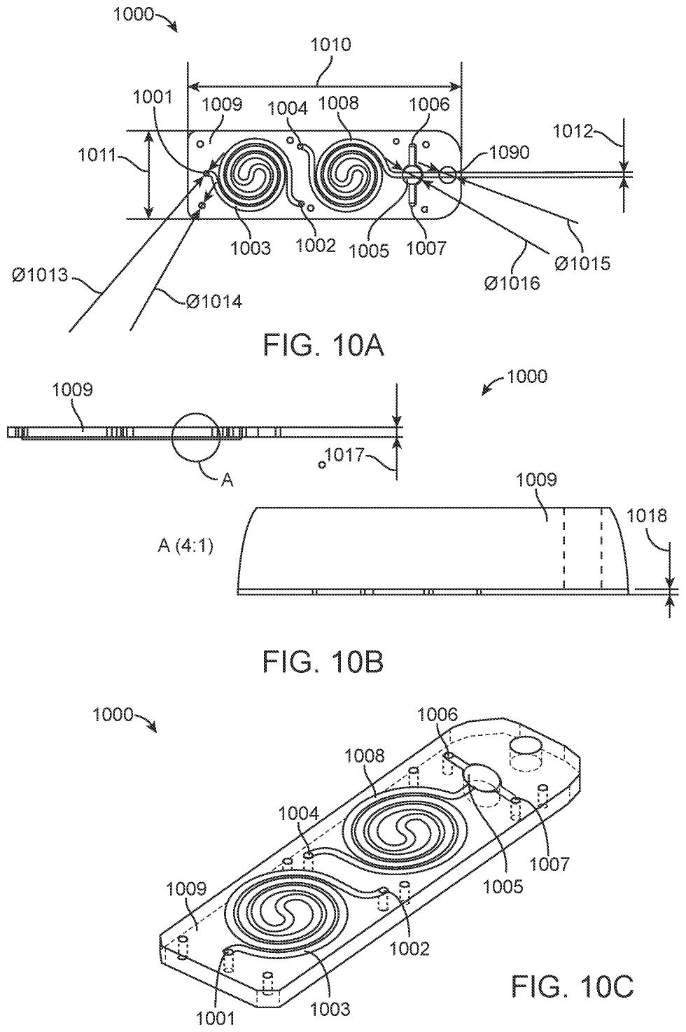

FIG. 10A, FIG. 10B, and FIG. 10C illustrate three views of top plate 1000 of the device 900. FIG. 10A illustrates a top view of top plate 1000, in accordance with some embodiments. FIG. 10B illustrates a side view of top plate 1000 with a 4× magnified inset, in accordance with some embodiments. FIG. 10C illustrates an isomorphic view of a top plate 1000, in accordance with some embodiments. Top plate 1000 and bottom plate 1100 may together form a top and a bottom portion of two multidirectional channels. Top plate 1000 and bottom plate 1100 may interlock to form multidirectional channels with a channel height of about 0.5 mm. The two parts may be separable to aid in cleaning; however, in some cases, the two parts may be sealed upon manufacturing. In some cases, a glue may seal top plate 1000 to bottom plate 1100.

As shown in FIG. 10A top plate 1000 may have a first inlet opening 1001 and a first outlet opening 1002 of a top of a first multidirectional channel 1003. Top plate 1000 may have a second inlet opening 1004 and a second outlet 1005 opening of a top of a second multidirectional channel 1008. The first 1003 and second 1008 channel of the multidirectional channel may comprise a groove milled into a substrate 1009. A second outlet 1005 may be connected to drain outlet 1007 and collection outlet 1006. A junction between the second outlet 1005, drain outlet 1007, and collection outlet 1006 may comprise a valve opening 1016. A valve opening may receive a neck of valve 1300. Top plate 1000 may additionally comprise one or more surface features 1015 to allow for storage and carrying of device 900.

As shown in FIG. 10A top plate 1000 may comprise width 1010, height 1011, and depth 1017. A width 1011 may comprise about 150 mm. A height 1011 may comprise about 50 mm. A depth 1017 may comprise about 6 mm. Top plate 1000 may comprise channel width 1012. Top plate 1000 may comprise tapped holes 1013 to allow luer lock fittings to be attached. Top plate 1000 may comprise thru-bores 1014 to allow bottom plate 1100 to be attached by screws or pins to top plate 1000.

FIG. 10B illustrates a side view of top plate 1000, in accordance with some embodiments. Substrate 1009 may comprise one or more thru-bore and/or openings. As shown in the inset, substrate 1009 may have grooves milled into a bottom surface with a depth 1018 to form a top of a multidirectional channel.

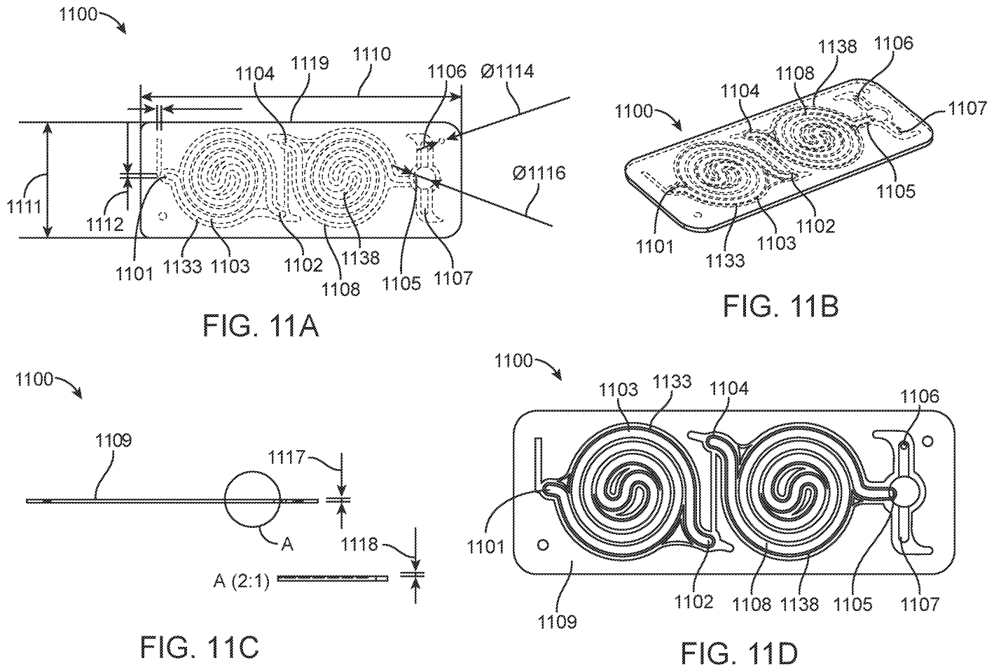

FIG. 11A, FIG. 11B, FIG. 11C, and FIG. 11D illustrate four views of bottom plate 1100 of the device 900. FIG. 11A illustrates a top view of bottom plate 1100, in accordance with some embodiments. FIG. 11B illustrates a side view of bottom plate 1100 with a 2× magnified inset, in accordance with some embodiments. FIG. 11C illustrates an isomorphic view of a bottom plate 1100, in accordance with some embodiments. FIG. 11D illustrates a top transparent view of a bottom plate 1100, in accordance with some embodiments.

As shown in FIG. 11A bottom plate 1100 may have a first inlet opening 1101 and a first outlet opening 1102 of a bottom of a first multidirectional channel 1103. Bottom plate 1100 may have a second inlet opening 1104 and a second outlet 1105 opening of a bottom of a second multidirectional channel 1108. The first 1103 and second 1108 channel of the multidirectional channel may comprise a groove milled into a substrate 1109. A second outlet 1105 may be connected to drain outlet 1107 and collection outlet 1106. A junction between the second outlet 1105, drain outlet 1107, and collection outlet 1106 may comprise a valve opening 1116. A valve opening may receive a base of valve 1300.

FIG. 11A also shows grooves 1133 and 1138 around the first and second multidirectional channels. Grooves 1133 and 1138 may be filed with a glue to permanently adhere the top and bottom plates. The grooves may be filled with a UV adhesive. The grooves may comprise a 1.91 mm width 1119 and a 0.5 mm height. In some examples, grooves 1133 and 1138 may be filed with a fluid such as saline which may provide removable seal to the first and second multidirectional channels. The channels and fluid may facilitate cleaning of the device for multiple reuses while providing a sterile seal.

As shown in FIG. 11A bottom plate 1100 may comprise width 1110, height 1111, and depth 1117. A width 1111 may comprise about 150 mm. A height 1111 may comprise about 50 mm. A depth 1117 may comprise about 2 mm. Bottom plate 1100 may comprise channel width 1112 which may be substantially equal to channel width 1012. Bottom plate 1100 may comprise attachment openings 1114 to allow top plate 1000 to be attached by screws or pins to bottom plate 1100.

FIG. 11B illustrates a side view of top plate 1100, in accordance with some embodiments. Substrate 1109 may comprise one or more thru-bores and/or openings. As shown in the inset, substrate 1109 may have grooves milled into a top surface with a depth 1118 to form a bottom of a multidirectional channel.

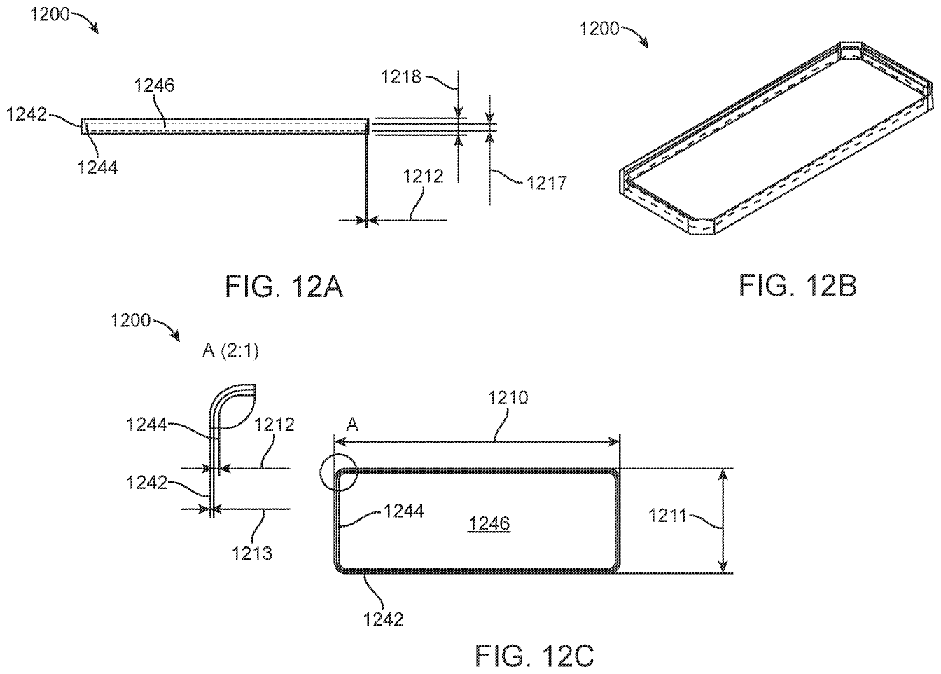

FIG. 12A, FIG. 12B, and FIG. 12C illustrate three views of reservoir 1200 of the device 900. FIG. 12A illustrates a side view of reservoir 1200, in accordance with some embodiments. FIG. 12B illustrates a top view of reservoir 1200 with a 4× magnified inset of a rim of reservoir 1200, in accordance with some embodiments. FIG. 12C illustrates an isomorphic view of reservoir 1200, in accordance with some embodiments.

As shown in FIG. 12A, reservoir 1200 may comprise and interior volume 1246 which may hold a volume of a sample. The reservoir volume may contain a waste sample. The reservoir 1200 may provide a sterile container for a sample. Reservoir 1200 may comprise interior surface 1244 and exterior surface 1242. A wall thickness of reservoir 1200 may be about 2.5 mm. Reservoir 1200 may comprise width 1210, height 1211, and depth 1218. A width 1211 may comprise about 150 mm. A height 1211 may comprise about 50 mm. A depth 1218 may comprise about 8 mm. An internal depth 1217 of reservoir 1200 may be about 5 mm.

As shown in the inset to FIG. 12C, a wall of reservoir 1200 may comprise a groove to receive top plate 1000 and bottom plate 1100. Dimensions 1212 and 1213 are shown. Dimension 1212 may be about 1.25 mm. Dimension 1213 may be about 1.25 mm. The groove may allow the bottom plate to snap fit into the reservoir.

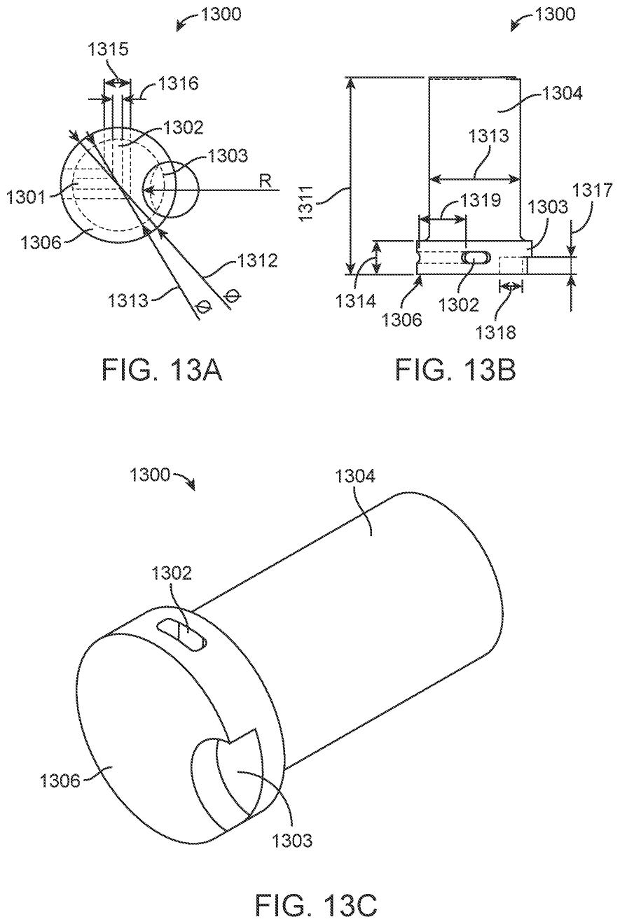

FIG. 13A, FIG. 13B, and FIG. 13C illustrate three views of valve 1300 of the device 900. FIG. 13A illustrates a bottom view of valve 1300, in accordance with some embodiments. FIG. 13B illustrates a side view of valve 1300, in accordance with some embodiments. FIG. 13C illustrates an isomorphic view of a top plate 1300, in accordance with some embodiments.

Valve 1300 comprises openings 1301 and 1302 connected by a channel. The openings intersect at about a 90 degree angle and may be positioned such that an outlet the second multidirectional channel may connected to either of a drain outlet or a collection outlet. Valve 1300 also comprises a stop 1303. The openings are disposed on a bottom portion of a valve which may be placed within the valve opening defined by 1016 and 1116. Valve 1300 also comprise a handle portion 1304 which may allow a user to orient the valve by rotation of the handle portion. A bottom surface 1306 may be in contact with a bottom plate.

Valve 1300 may comprise height 1311, bottom diameter 1312, top diameter 1313, bottom portion height 1314, channel width 1315, channel height 1316, and stop height 1317 and depth 1318. Height 1311 may comprise about 20 mm. Bottom diameter 1312 may comprise about 10 mm. Top diameter 1313 may comprise about 8 mm. Bottom portion height 1314 may comprise about 3 mm. Channel width 1315 may comprise about 2.5 mm. Channel height 1316 may comprise about 1 mm. Stop height 1317 may comprise about 1.5 mm, and stop depth 1318 may comprise about 2 mm.

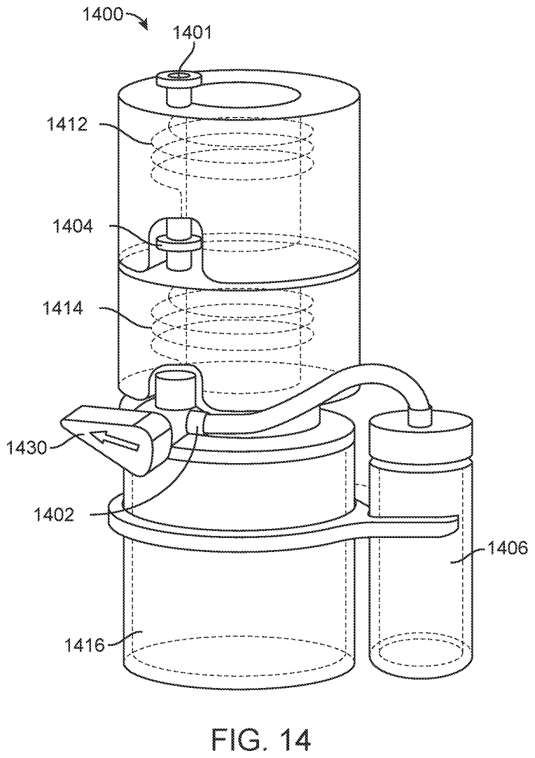

FIG. 14 illustrates a non-limiting example of a device 1400 comprising a multidirectional channel. Device 1400 may comprise inlet 1401 and outlet 1402. Inlet 1401 provides access to first multidirectional channel 1412. A sample may exit first multidirectional channel 1412 at junction 1404. Junction 1404 may provide access to second multidirectional channel 1414. A sample may exit second multidirectional channel 1414 at outlet 1402. Outlet 1402 may fluidically connected to valve 1430. Valve 1403 may control flow to a reservoir 1416 or a collection tube 1406.

Due to the vertical stacking of the channels, flow in device 1400 may be induced by force of gravity. In some cases, a sample may be pressed through the channel using a luer lock syringe. Inlet 1401 may comprise a luer lock fitting. The channel of the fluidic cartridge is then be coated with a substance, such as polymyxin or vancomycin, using previously described methods. The coating should coat at least a portion of an inside wall of the multidirectional channel(s). In some cases, a first multidirectional channel may comprise colistin and a second may comprise vancomycin. In some cases, a first multidirectional channel may comprise vancomycin and a second may comprise colistin. The colistin and vancomycin may be added as described herein, for example, with respect to FIG. 3 and FIG. 4.

The device 1400 may be manufactured through any number of technologies. For example, among the more common methods of manufacture are 3-D printing, stereolithography, photolithography, injection molding, blow molding, casting, ultrasonic welding, high frequency welding, heated tool or plate welding, solvent bonding, laser welding, spin welding, infrared welding, vibration welding, adhesive bonding, and machining. In some embodiments, the preferred machining methods are turning, drilling, boring, reaming, electric discharge machining (EDM) and/or milling. Material are commonly machined to create an enclosure of desired dimensions, such as by machining channel(s) as a whole or by machining halves that are then sealed and enclosed using bolts, adhesive, thermal expansion, or another means. The machining may be performed on various biocompatible materials, including various grades of polycarbonate.

Systems and methods of the present disclosure may comprise other geometries of devices such as those disclosed in commonly owned P.C.T. Application No. PCT/US2019/012403, which is incorporated herein by reference in its entirety.

Systems and methods of the present disclosure are used for isolation of pathogens from fluids for diagnosis of infection, collection and concentration of pathogens, transfusion medicine, sepsis treatment, bacteremia treatment, endotoxemia treatment, and other blood-borne diseases. These fluids, such as blood, would then be passed or circulated through the channel(s) of the bacterial and endotoxin adsorbent fluidic cartridge.

A method of the present disclosure also includes a method in which thermoplastic tubing is coated with the polypeptide antibiotic substance.

In a preferable mode, bacteria are isolated from a sample of blood wherein the blood sample is passed through the multidirectional bacterial adsorbent fluidic cartridge of the device. The bacteria captured within the channel are eluted and lysed using the elution/lysis buffer, which then allows for collection of bacterial DNA for rapid testing (as illustrated in non-limiting FIG. 2). In one example, the fluidic cartridge is functionalized along the channel walls with polypeptide antibiotics designed to capture bacteria and endotoxin. Following processing through the fluidic cartridge, the elution/lysis buffer is then moved through the fluidic cartridge using a syringe. The eluate is collected from the device outlet and analyzed.

The elution/lysis buffer, also referred to as an “eluant” herein, may comprise an anticoagulant, a lysing agent, a protease, one or more denaturing agents, and one or more buffering agents. The eluant may comprise EDTA (Ethylenediaminetetraacetic acid), Triton X-100, Tris-HCl (2-Amino-2-(hydroxymethyl)-1,3-propanediol hydrochloride), Proteinase K, GuSCN (Guanidine thiocyanate), SDS (Dodecyl sodium sulfate), and distilled water (DiH2O). The solution may be vortexed to mix and sonicated. The solution may be homogenous.

In some embodiments of the method, the eluant comprises: about 2 mM EDTA (Ethylenediaminetetraacetic acid), about 2.5% (v/v) Triton X-100, about 20 mM Tris-HCl pH 8.0 (2-Amino-2-(hydroxymethyl)-1,3-propanediol hydrochloride), about 0.03 mM Proteinase K, about 2.75M GuSCN (Guanidine thiocyanate), about 0.04M SDS (Dodecyl sodium sulfate) and distilled water (DiH2O). The term “about” when used in reference to the amount of a constituent of an eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range.

In a preferable mode, the elution/lysis buffer comprises at least one of Tris-HCl, EDTA, sodium dodecyl sulfate (SDS), TritonX-100, chaotropic buffer, and/or proteinase K; sodium heparin; a heparin compound; fluoride; oxalate; sodium citrate; sodium polyanethol sulfonate (SPS); Acid Citrate Dextrose Solution (sometimes called Anticoagulant Citrate Dextrose Solution); distilled H₂O; or saline.

In some embodiments of the method, the eluant is selected from a group comprising: Tris-HCl, Ethylenediaminetetraacetic acid (EDTA), sodium dodecyl sulfate (SDS), TritonX-100, a chaotropic buffer or proteinase K. In some embodiments of the method, the eluant contains between 0.001M to about 100 M of Tris-HCL. In some embodiments of the method, the eluant contains between 0.001M to about 100M of EDTA. In some embodiments, the eluant has a pH between a range of 2 and 13. In preferred embodiments the eluant has a pH between a range of 6 and 9.

In some embodiments of the method, the eluant contains an anticoagulant. The anticoagulant may comprise EDTA. The anticoagulant may comprise EDTA, citrate, oxalate, and/or fluoride. The anticoagulant may comprise heparin, warfarin, rivaroxaban, dabigatran, apixaban, edoxaban, enoxaparin, fondaparinux, or similar. The eluant may comprise EDTA at a concentration of about 2 mM. The eluant may comprise an anticoagulant at a concentration between about 0.001 M to about 100 M. In some embodiments of the method, the eluant contains an anticoagulant at a concentration between at least about 0.001 M. In some embodiments of the method, the eluant contains an anticoagulant at a concentration between at most about 100 M. In some embodiments of the method, the eluant contains an anticoagulant at a concentration between about 0.001 M to about 0.01 M, about 0.001 M to about 0.1 M, about 0.001 M to about 1 M, about 0.001 M to about 10 M, about 0.001 M to about 25 M, about 0.001 M to about 50 M, about 0.001 M to about 75 M, about 0.001 M to about 100 M, about 0.01 M to about 0.1 M, about 0.01 M to about 1 M, about 0.01 M to about 10 M, about 0.01 M to about 25 M, about 0.01 M to about 50 M, about 0.01 M to about 75 M, about 0.01 M to about 100 M, about 0.1 M to about 1 M, about 0.1 M to about 10 M, about 0.1 M to about 25 M, about 0.1 M to about 50 M, about 0.1 M to about 75 M, about 0.1 M to about 100 M, about 1 M to about 10 M, about 1 M to about 25 M, about 1 M to about 50 M, about 1 M to about 75 M, about 1 M to about 100 M, about 10 M to about 25 M, about 10 M to about 50 M, about 10 M to about 75 M, about 10 M to about 100 M, about 25 M to about 50 M, about 25 M to about 75 M, about 25 M to about 100 M, about 50 M to about 75 M, about 50 M to about 100 M, or about 75 M to about 100 M. The term “about” when used in reference to the amount an anticoagulant of eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments of the method, the eluant contains a lysing agent. The lysing agent may comprise a detergent. The lysing agent may comprise one or more of Triton X-100, Triton X-114, NP-40, Brij-35, Brij-58, Tween 20, Tween 80, Octyl glucoside, Octyl thioglucoside, SDS, CHAPS, CHAPSO, or similar. For example, the lysing agent may comprise Triton X-100. The lysing agent may comprise Trion X-100 at a concentration of about 2.5% (v/v). The eluant may comprise a lysing agent at a concentration between about 0.001% (v/v) to about 50% (v/v). In some embodiments of the method, the eluant contains a lysing agent at a concentration between at least about 0.001% (v/v). In some embodiments of the method, the eluant contains a lysing agent at a concentration at most about 50% (v/v). In some embodiments of the method, the eluant contains a lysing agent at a concentration between about 0.001% (v/v) to about 0.01% (v/v), about 0.001% (v/v) to about 0.1% (v/v), about 0.001% (v/v) to about 1% (v/v), about 0.001% (v/v) to about 10% (v/v), about 0.001% (v/v) to about 25% (v/v), about 0.001% (v/v) to about 50% (v/v), about 0.01% (v/v) to about 0.1% (v/v), about 0.01% (v/v) to about 1% (v/v), about 0.01% (v/v) to about 10% (v/v), about 0.01% (v/v) to about 25% (v/v), about 0.01% (v/v) to about 50% (v/v), about 0.1% (v/v) to about 1% (v/v), about 0.1% (v/v) to about 10% (v/v), about 0.1% (v/v) to about 25% (v/v), about 0.1% (v/v) to about 50% (v/v), about 1% (v/v) to about 10% (v/v), about 1% (v/v) to about 25% (v/v), about 1% (v/v) to about 50% (v/v), about 10% (v/v) to about 25% (v/v), about 10% (v/v) to about 50% (v/v), about 25% (v/v) to about 50% (v/v). The term “about” when used in reference to the amount a lysing agent of an eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments of the method, the eluant contains a protease. The protease may comprise a serine protease. The protease may comprise one or more of Endoproteinase, Trypsin, Chymotrypsin, Endoproteinase Asp-N, Endoproteinase Arg-C, Endoproteinase Glu-C, Endoproteinase Lys-C, Pepsin, Thermolysin, Elastase, Papain, Proteinase K, Subtilisin, Clostripain, Exopeptidase, Carboxypeptidase A, Carboxypeptidase B, Carboxypeptidase P, Carboxypeptidase Y, Cathepsin C, Acylamino-acid-releasing enzyme, Pyroglutamate aminopeptidase, or combinations thereof. The protease may comprise Proteinase K. The protease may comprise Proteinase K at a concentration of 0.03 mM. The eluant may comprise a protease at a concentration between about 0.001 M to about 100 M. In some embodiments of the method, the eluant contains a protease at a concentration between at least about 0.001 M. In some embodiments of the method, the eluant contains a protease at a concentration between at most about 100 M. In some embodiments of the method, the eluant contains a protease at a concentration between about 0.001 M to about 0.01 M, about 0.001 M to about 0.1 M, about 0.001 M to about 1 M, about 0.001 M to about 10 M, about 0.001 M to about 25 M, about 0.001 M to about 50 M, about 0.001 M to about 75 M, about 0.001 M to about 100 M, about 0.01 M to about 0.1 M, about 0.01 M to about 1 M, about 0.01 M to about 10 M, about 0.01 M to about 25 M, about 0.01 M to about 50 M, about 0.01 M to about 75 M, about 0.01 M to about 100 M, about 0.1 M to about 1 M, about 0.1 M to about 10 M, about 0.1 M to about 25 M, about 0.1 M to about 50 M, about 0.1 M to about 75 M, about 0.1 M to about 100 M, about 1 M to about 10 M, about 1 M to about 25 M, about 1 M to about 50 M, about 1 M to about 75 M, about 1 M to about 100 M, about 10 M to about 25 M, about 10 M to about 50 M, about 10 M to about 75 M, about 10 M to about 100 M, about 25 M to about 50 M, about 25 M to about 75 M, about 25 M to about 100 M, about 50 M to about 75 M, about 50 M to about 100 M, or about 75 M to about 100 M. The term “about” when used in reference to the amount a protease of an eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments of the method, the eluant comprises one or more denaturing agents. The denaturing agent may comprise one or more of formamide, guanidine, sodium salicylate, dimethyl sulfoxide (DMSO), propylene glycol, urea, and combinations thereof. Other chemical denaturing agents may comprise acids, alkalis, heavy metal salts, alcohols such as ethanol, etc. For example, the denaturing agent may comprise guanidine thiocyanate. The denaturing agent may comprise guanidine thiocyanate a concentration of about 2.75M. The eluant may comprise a denaturing agent at a concentration between about 0.001 M to about 100 M. In some embodiments of the method, the eluant contains a denaturing agent at a concentration between at least about 0.001 M. In some embodiments of the method, the eluant contains a denaturing agent at a concentration between at most about 100 M. In some embodiments of the method, the eluant contains a denaturing agent at a concentration between about 0.001 M to about 0.01 M, about 0.001 M to about 0.1 M, about 0.001 M to about 1 M, about 0.001 M to about 10 M, about 0.001 M to about 25 M, about 0.001 M to about 50 M, about 0.001 M to about 75 M, about 0.001 M to about 100 M, about 0.01 M to about 0.1 M, about 0.01 M to about 1 M, about 0.01 M to about 10 M, about 0.01 M to about 25 M, about 0.01 M to about 50 M, about 0.01 M to about 75 M, about 0.01 M to about 100 M, about 0.1 M to about 1 M, about 0.1 M to about 10 M, about 0.1 M to about 25 M, about 0.1 M to about 50 M, about 0.1 M to about 75 M, about 0.1 M to about 100 M, about 1 M to about 10 M, about 1 M to about 25 M, about 1 M to about 50 M, about 1 M to about 75 M, about 1 M to about 100 M, about 10 M to about 25 M, about 10 M to about 50 M, about 10 M to about 75 M, about 10 M to about 100 M, about 25 M to about 50 M, about 25 M to about 75 M, about 25 M to about 100 M, about 50 M to about 75 M, about 50 M to about 100 M, or about 75 M to about 100 M. The term “about” when used in reference to the amount a denaturing agent of an eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the eluant comprises a buffering agent. The buffering agent may comprise one or more of Tris buffers (Tris-HCl, Tris-EDTA, Tris-SSC, SSPE, Tris-TE, Tris-STE), Tris-HCl, glycine-HCl, glycine-NaOH, sodium cacodylate, tromethamine, potassium phosphate, sodium phosphate, saline sodium citrate buffer (SSC), acetate, saline, physiological saline, buffered saline (PBS, TBS, TNT, PBT), phosphate buffer saline (PBS), 4-2-hydroxyethyl-1-piperazineethanesulfonic acid buffer (HEPES), 3-(N-morpholino)propanesulfonic acid buffer (MOPS), and piperazine-N,N′-bis(2-ethanesulfonic acid) buffer (PIPES), sodium acetate-boric acid stock solution, boric acid-sodium carbonate with sodium chloride solution, boric acid-sodium borate buffer, sodium and potassium phosphate buffers, boric acid-sodium carbonate with potassium chloride, or combinations thereof. The buffer may comprise Tris-HCl pH 8.0 (2-Amino-2-(hydroxymethyl)-1,3-propanediol hydrochloride). The buffer may comprise SDS (Dodecyl sodium sulfate). The buffer may comprise Tris-HCl pH 8.0 (2-Amino-2-(hydroxymethyl)-1,3-propanediol hydrochloride) at a concentration of about 20 mM and 0.04M SDS (Dodecyl sodium sulfate). The eluant may comprise an anticoagulant at a concentration between about 0.001 M to about 100 M. In some embodiments of the method, the eluant contains a buffering agent at a concentration between at least about 0.001 M. In some embodiments of the method, the eluant contains a buffering agent at a concentration between at most about 100 M. In some embodiments of the method, the eluant contains a buffering agent at a concentration between about 0.001 M to about 0.01 M, about 0.001 M to about 0.1 M, about 0.001 M to about 1 M, about 0.001 M to about 10 M, about 0.001 M to about 25 M, about 0.001 M to about 50 M, about 0.001 M to about 75 M, about 0.001 M to about 100 M, about 0.01 M to about 0.1 M, about 0.01 M to about 1 M, about 0.01 M to about 10 M, about 0.01 M to about 25 M, about 0.01 M to about 50 M, about 0.01 M to about 75 M, about 0.01 M to about 100 M, about 0.1 M to about 1 M, about 0.1 M to about 10 M, about 0.1 M to about 25 M, about 0.1 M to about 50 M, about 0.1 M to about 75 M, about 0.1 M to about 100 M, about 1 M to about 10 M, about 1 M to about 25 M, about 1 M to about 50 M, about 1 M to about 75 M, about 1 M to about 100 M, about 10 M to about 25 M, about 10 M to about 50 M, about 10 M to about 75 M, about 10 M to about 100 M, about 25 M to about 50 M, about 25 M to about 75 M, about 25 M to about 100 M, about 50 M to about 75 M, about 50 M to about 100 M, or about 75 M to about 100 M. The term “about” when used in reference to the amount a buffering agent of eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” means within 30.0 mM, 20.0 mM, 10.0 mM, 5.0 mM, 1.0 mM, 0.9 mM, 0.8 mM, 0.7 mM, 0.6 mM, 0.5 mM, 0.4 mM, 0.3 mM, 0.2 mM, 0.1 mM, 0.09 mM, 0.08 mM, 0.07 mM, 0.06 mM, 0.05 mM, 0.04 mM, 0.03 mM, 0.02 mM or 0.01 mM of a given value or range.

In some embodiments, the eluant has a pH of about 2 to about 13. In some embodiments, the eluant has a pH of at least about 2. In some embodiments, the eluant has a pH of at most about 13. In some embodiments, the eluant has a pH of about 2 to about 3, about 2 to about 4, about 2 to about 5, about 2 to about 6, about 2 to about 7, about 2 to about 8, about 2 to about 9, about 2 to about 10, about 2 to about 11, about 2 to about 12, about 2 to about 13, about 3 to about 4, about 3 to about 5, about 3 to about 6, about 3 to about 7, about 3 to about 8, about 3 to about 9, about 3 to about 10, about 3 to about 11, about 3 to about 12, about 3 to about 13, about 4 to about 5, about 4 to about 6, about 4 to about 7, about 4 to about 8, about 4 to about 9, about 4 to about 10, about 4 to about 11, about 4 to about 12, about 4 to about 13, about 5 to about 6, about 5 to about 7, about 5 to about 8, about 5 to about 9, about 5 to about 10, about 5 to about 11, about 5 to about 12, about 5 to about 13, about 6 to about 7, about 6 to about 8, about 6 to about 9, about 6 to about 10, about 6 to about 11, about 6 to about 12, about 6 to about 13, about 7 to about 8, about 7 to about 9, about 7 to about 10, about 7 to about 11, about 7 to about 12, about 7 to about 13, about 8 to about 9, about 8 to about 10, about 8 to about 11, about 8 to about 12, about 8 to about 13, about 9 to about 10, about 9 to about 11, about 9 to about 12, about 9 to about 13, about 10 to about 11, about 10 to about 12, about 10 to about 13, about 11 to about 12, about 11 to about 13, or about 12 to about 13.

In some preferred embodiments, the eluant has a pH of about 6 to about 9. In some preferred embodiments, the eluant has a pH of at least about 6. In some preferred embodiments, the eluant has a pH of at most about 9. In some preferred embodiments, the eluant has a pH of about 6 to about 7, about 6 to about 8, about 6 to about 9, about 7 to about 8, about 7 to about 9, or about 8 to about 9.

Provided herein is a method for isolation of materials of interest comprising: bringing a sample of fluid in contact with a multidirectional, polypeptide antibiotic coated channel; adsorbing (capturing) said material(s) of interest on said multidirectional channel walls; eluting and lysing said captured materials of interest with an elution/lysis buffer; and analyzing the presence or amount of said materials of interest eluted from the multidirectional, polypeptide antibiotic coated channel.

In some embodiments of the method, a fluid sample is brought in contact with a multidirectional, polypeptide antibiotic coated channel using a syringe. In some embodiments of the method, the syringe is a luer lock syringe. In some embodiments of the method, a fluid sample is brought in contact with a multidirectional, polypeptide antibiotic coated channel using a pump. In some embodiments of the method, following adsorbing said material(s) of interest on said multidirectional channel walls, an elution/lysis buffer is brought in contact with a multidirectional, polypeptide antibiotic coated channel using a syringe. In some embodiments, the syringe is a luer lock syringe.

In some embodiments of the method the elution/lysis buffer, (alternatively called the eluant, herein) is selected from a group comprising: a Tris-HCl; an Ethylenediaminetetraacetic acid (EDTA); a sodium dodecyl sulfate (SDS); a TritonX-100; a chaotropic buffer; or a proteinase K; sodium heparin; a heparin compound; fluoride; oxalate; sodium citrate; sodium polyanethol sulfonate (SPS); Acid Citrate Dextrose Solution (sometimes called Anticoagulant Citrate Dextrose Solution); distilled H₂O; or saline.

In some embodiments of the method, the eluant is selected from a group comprising: Tris-HCl, Ethylenediaminetetraacetic acid (EDTA), sodium dodecyl sulfate (SDS), TritonX-100, a chaotropic buffer or proteinase K. In some embodiments of the method, the eluant contains between 0.001M to about 100 M of Tris-HCL. In some embodiments of the method, the eluant contains between 0.001M to about 100M of EDTA. In some embodiments, the eluant has a pH between a range of 2 and 13. In preferred embodiments the eluant has a pH between a range of 6 and 9. In some embodiments of the method, the elution buffer contains between 0.01% and about 90% of sodium dodecyl sulfate having a pH between 6 and 9. In some embodiments of the method, the elution buffer contains between 0.01% and about 90% of TritonX-100 having a pH between 6 and 9. In some embodiments of the method, the elution buffer contains between 0.001M and about 100M of a chaotropic buffer having a pH between 6 and 9. In some embodiments of the method, the elution buffer contains the chaotropic buffer Guanidinium thiocyanate having a pH between 6 and 9. In some embodiments of the method, the elution buffer contains between 0.001 mg/mL and about 100 mg/mL of proteinase K having a pH between 6 and 9.

In some embodiments of the method, the eluant comprises: about 2 mM EDTA (Ethylenediaminetetraacetic acid), about 2.5% (v/v) Triton X-100, about 20 mM Tris-HCl pH 8.0 (2-Amino-2-(hydroxymethyl)-1,3-propanediol hydrochloride), about 0.03 mM Proteinase K, about 2.75M GuSCN (Guanidine thiocyanate), about 0.04M SDS (Dodecyl sodium sulfate) and distilled water (DiH2O). The term “about” when used in reference to the amount of a constituent of an eluant, in some embodiments means within 30%, 25%, 20%, 15%, 10%, 9%, 8%, 7%, 6%, 5%, 4%, 3%, 2%, 1%, 0.5%, 0.1%, or 0.05% of a given value or range. Further examples and embodiments of an eluant are described elsewhere herein.

In some embodiments of the method, the eluted material of interest, alternatively called, the “eluted target(s) of interest,” “eluted disease-causing material(s),” “eluted materials of interest,” or “eluted disease material,” used synonymously herein, is separated from the eluant, concentrated and washed. In some embodiments of the method, the eluant of interest retained from the fluidic cartridge outlet is washed using spin filtration.

In some embodiments of the method, detection of the presence or amount of materials of interest captured within the multidirectional, polypeptide antibiotic coated channel is performed by analyzing the materials of interest following elution from the fluidic cartridge using said elution/lysis buffer. In some embodiments, an analysis method is selected from the group comprising: cell counting, MALDI-TOF MS (matrix assisted laser desorption ionization-time of flight mass spectrometry), mass spectrometry, PCR (polymerase chain reaction), biosensing, flow cytometry, and fluorescent labeling. In some embodiments, the eluted material of interest is analyzed using at least one of: a polymerase chain reaction (PCR), a matrix-assisted laser desorption/ionization-time of flight, (MALDI-TOF), a nuclear magnetic resonance (NMR) spectroscopy, culturing, a fluorescence in situ hybridization (FISH), optically active microbeads and optically active nanoparticles.

Provided herein is a method of isolating one or more target(s) of interest from a sample of flowing matter using fluidic devices that detect, capture, adsorb, and/or remove the target(s) of interest (alternatively called “disease-causing material(s)” or “material(s) of interest”) from biological fluids and the subsequent diagnosis and treatment of disease based thereon. The method comprises; (1) flowing the sample through a multidirectional fluidic cartridge channel that is coated with a substance, causing the sample to come in contact with the coating substance, thereby forming a bond between said substance and said the materials of interest within the sample; (2) separating the bound materials of interest from sample matter that is not of interest by washing the multidirectional fluidic cartridge channel with a wash buffer and then discarding said wash buffer containing the residual sample matter that is not of interest; (3) collecting the materials of interest by flowing an elution buffer (eluant) through the multidirectional fluidic cartridge channel, thus concentrating the materials of interest in an eluate; and (4) the eluate containing the concentrated materials of interest is purified by introducing the eluate into a spin column. The spin column is placed in a centrifuge for centrifugation. During centrifugation, target sample materials are entrapped in the spin column matrix, while other unwanted materials pass through the spin column matrix into the collection vial. The collection vial is discarded. The targeted, isolated sample material entrapped in the spin column matrix is then released from the matrix and collected into a clean collection vial by washing the matrix with ethyl alcohol. In some embodiments, the collected isolated materials of interest are analyzed. Analysis of the isolated materials of interest comprises a polymerase chain reaction (PCR)-based assay, a nuclear magnetic resonance (NMR) spectroscopy, a matrix-assisted laser desorption/ionization-time of flight (MALDI-TOF), a fluorescence in situ hybridization (FISH), an immunoassay, an enzyme-linked immunosorbent assay (ELISA), optically active microbeads, optically active nanoparticles, a culturing assay with optional staining, or a combination thereof.

In some embodiments, the sample is selected from the group consisting of a blood, a serum, a plasma, a urine, a cerebral spinal fluid, a bronchial fluid, another bodily fluid, a homogenized tissue, a homogenized food sample, and a combination thereof.

In some embodiments, the wash buffer comprises a saline, an ethanol, a sterile H₂O, or a combination thereof.

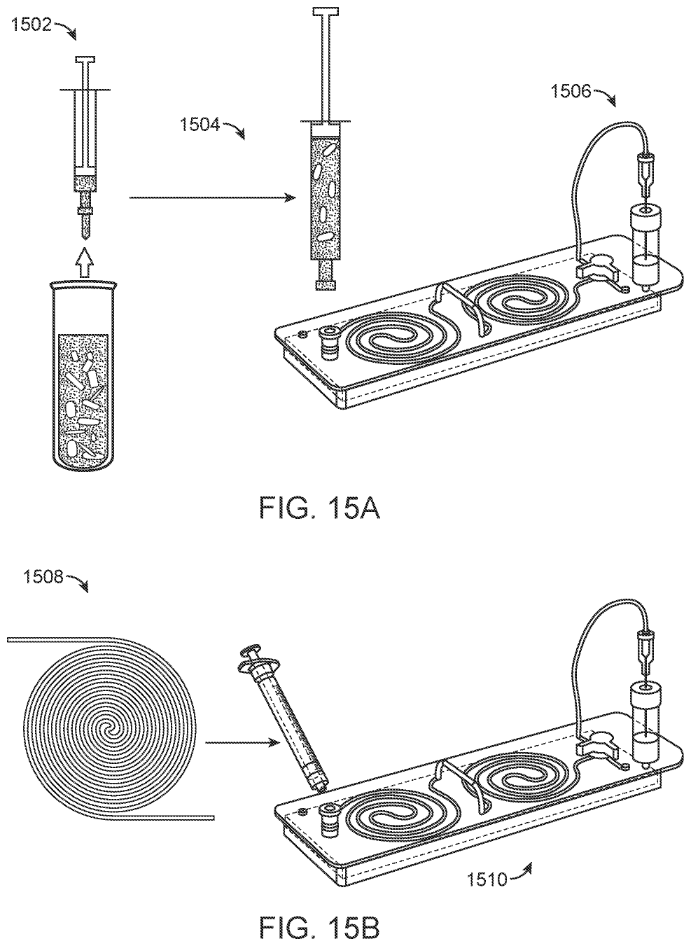

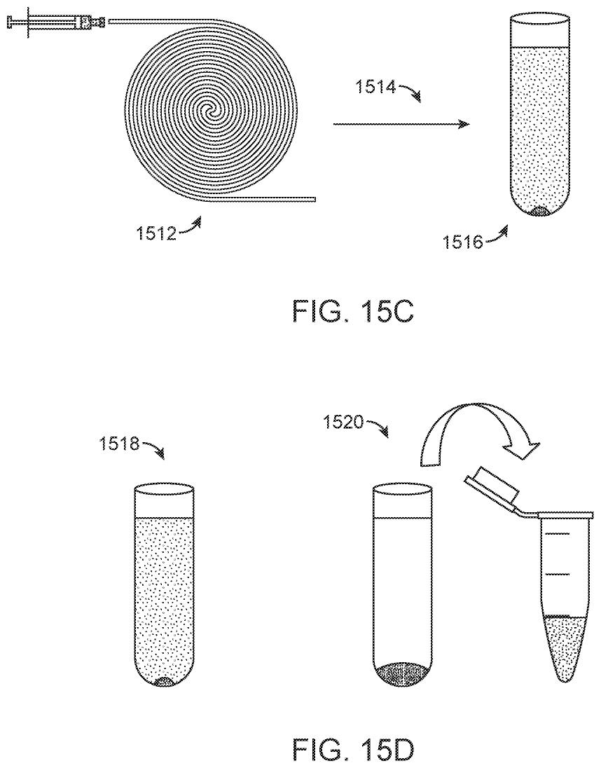

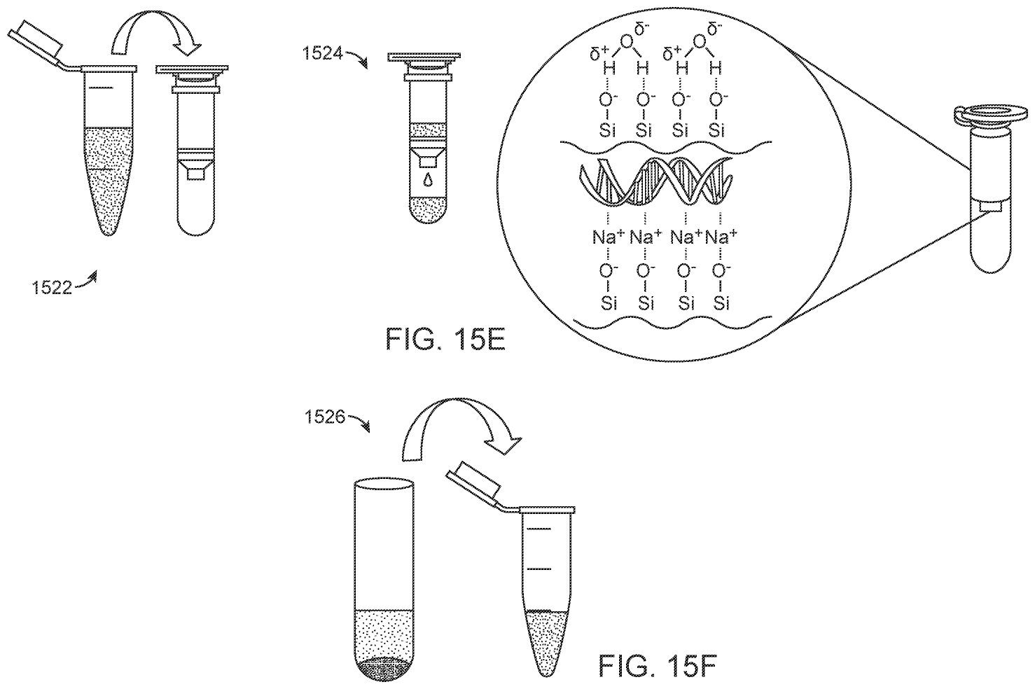

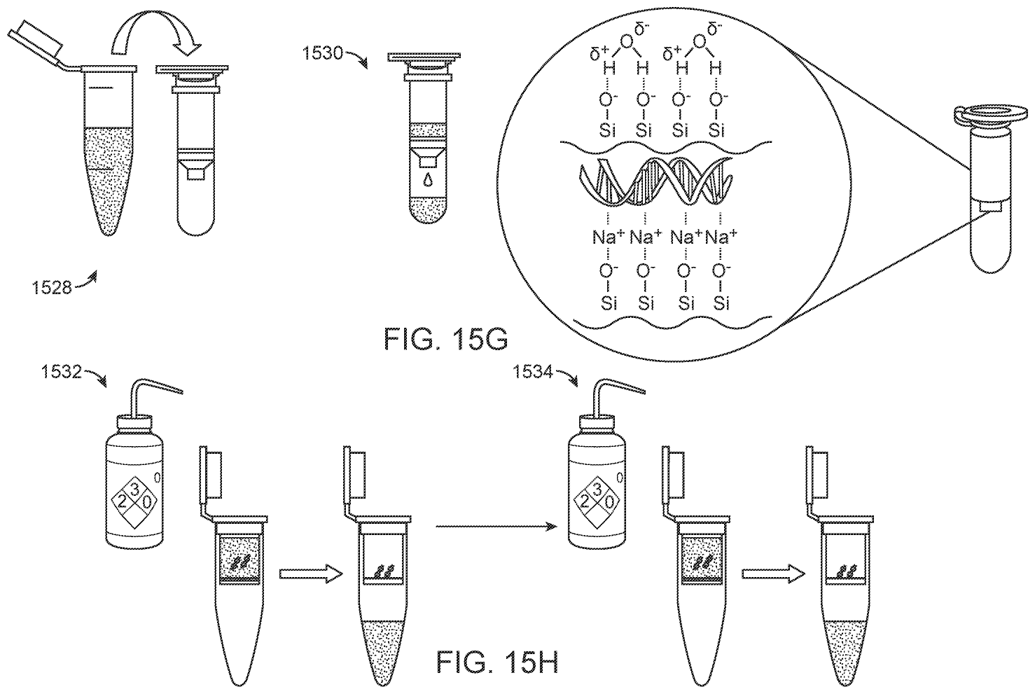

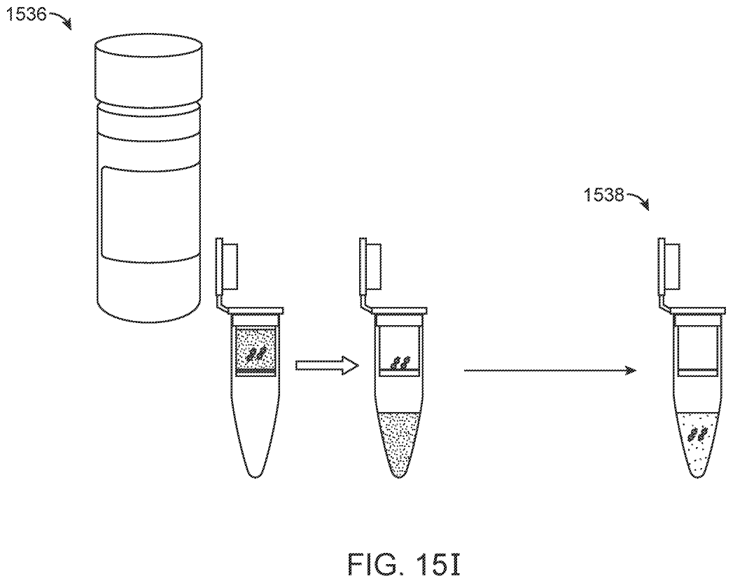

FIGS. 15A-15I illustrate a method 1500 of isolating one or more target(s) of interest from a sample of flowing matter. As shown in FIG. 15A, at an operation 1502 of the method 1500, a sample of a bodily fluid of a subject may be provided. A sample may comprise a blood, a serum, a plasma, a urine, a cerebral spinal fluid, a bronchial fluid, another bodily fluid, a homogenized tissue, a homogenized food sample, and a combination thereof. A sample may have a volume of less than 1 L, 100 mL, 50 mL, 10 mL, 1 mL, or less. A sample comprise about 10 mL of blood. A sample of blood may be whole blood. At an operation 1504 of the method 1500, a needle, if present may be removed and discarded. At an operation 1506 of the method 1500, a syringe may be attached to an example, embodiment, or variation of a device described herein. For example, a syringe may be attached to device 700, 800, 900, and/or 1400. FIG. 15A illustrates a device 900 as disclosed herein; however, other devices may be used.

As shown in FIG. 15B, at an operation 1508 of the method 1500 a sample may be injected into a multidirectional channel of the device. Depending on the design of the device, the sample may be pressed through the device by force of a plunger on the syringe, flow by force of gravity through the channel, be pumped by an external pressure, etc. The sample may flow through the multidirectional channel and one or more materials of interest may be absorbed on at least one wall of the multi-directional fluidic channel. Optionally, a valve may be oriented such that the sample may flow into a reservoir. After passing through the multidirectional channel, the one or more materials of interest may be substantially removed from the sample.

At an operation 1510 of the method 1500, a wash may be flowed through the multidirectional channel. An operation 1510 may separate the bound materials of interest from sample matter that may not of be of interest. A wash may comprise water. For example, wash water may be PCR grade water. A wash may have a volume of less than 50 mL, 10 mL, 1 mL, or less. A wash may comprise about 1 mL of water. Optionally, a wash may be exit into a reservoir, such as a waste container.

As shown in FIG. 15C, at an operation 1512 of the method 1500, an elution buffer may be injected into a multidirectional channel of the device. An elution buffer (eluant) may concentrate one or more materials of interest in an eluate. Optionally, at an operation 1514 of the method 1500, the valve may be oriented to a closed position and an elution buffer may be incubated in the multidirectional channel. An incubation time may be greater than 1 minute, 10 minutes, 1 hour, or more. An incubation time may be less than 10 hours, less than 1 hours, less than 10 minutes, or less. An incubation time may be about 20 minutes. At an operation 1516 of the method 1500, an eluate may be collected. The eluate may be collected by pushing additional eluate, water, or air through the multidirectional channel. The eluate may be collected by allowing the eluate to flow out of the multidirectional channel by force of gravity. The eluate may comprise a suspension of materials of interest and other sample particles.

After collection of the eluate one or more operations may be performed to extract and identify the material of interest. For example, a microbial species, virulence gene, or antibiotic resistance gene may be identified by a PCR assay. Analysis of the isolated materials of interest comprises a polymerase chain reaction (PCR)-based assay, a nuclear magnetic resonance (NMR) spectroscopy, a matrix-assisted laser desorption/ionization-time of flight (MALDI-TOF), a fluorescence in situ hybridization (FISH), an immunoassay, an enzyme-linked immunosorbent assay (ELISA), optically active microbeads, optically active nanoparticles, a culturing assay with optional staining, or a combination thereof.

Operations 1518 to 1538 provide an example of extracting and identifying a material of interest. Operations 1518 to 1538 directed to a species identification PCR assay.

As shown in FIG. 15D, at an operation 1518 of the method 1500, the eluate may be centrifuged. At an operation 1520 of the method 1500, all or a portion of the supernate may be collected. Optionally, the supernate may be added to a solvent. For example, the solvent may be an alcohol, such as ethanol.

As shown in FIG. 15E, at an operation 1522 of the method 1500, the supernate-solvent mixture may be added to a spin column. In some cases, the eluate may be directly added to a spin column. For example, the spin column may comprise a silica membrane spin column. At an operation 1524 of the method 1500, the spin column may be placed in a centrifuge for centrifugation. During centrifugation, target sample materials may be entrapped in the spin column matrix, while other unwanted materials may pass through the spin column matrix into the collection vial.

As shown in FIG. 15F, at an operation 1526 of the method 1500, if a portion rather than all of the supernate was collected. Further fractions of the supernate may be collected. Optionally, the supernate may be added to a solvent. For example, the solvent may be an alcohol, such as ethanol.

As shown in FIG. 15G, at an operation 1528 of the method 1500, the further supernate-solvent mixture may be added to a spin column. For example, the spin column may comprise a silica membrane spin column. In some cases, the spin column may be the spin column of operation 1522 with a new spin basket. At an operation 1530 of the method 1500, the spin column may be placed in a centrifuge for centrifugation. During centrifugation, further target sample materials may be entrapped in the spin column matrix, while other unwanted materials may pass through the spin column matrix into the collection vial.

As shown in FIG. 15H, at an operation 1532 of the method 1500, one or more solvent wash steps may be performed. For example, the contents of one or more previous spin columns may be added to a new spin column with an amount of solvent. For example, the contents of one or more previous spin columns may be added to the same spin column with a new spin basket and an amount of solvent. The solvent may be the same or different than the solvent of steps 1522 to 1530. The solvent may comprise an alcohol. The solvent may comprise water. The solvent may comprise an ethanol-water mixture. The contents may be centrifuged. At an operation 1534 of the method 1500, the operation 1532 may be repeated. The wash solvent of operation 1534 may be the same or different than the wash of operation 1532.

As shown in FIG. 15I, at an operation 1536 of the method 1500, the extracted DNA may be eluted. For example, the contents of one or more previous spin columns may be washed with solvent configured to elute the DNA of the material of interest. After a spin step, the DNA may be transferred to a PCR system for analysis.

Provided herein is a kit for the capture and adsorption of materials of interest comprising: a fluidic cartridge with an inlet and an outlet; a multidirectional fluidic channel between the inlet and the outlet; said multidirectional fluidic channel comprising: at least one wall; a substance coating an inside wall of the multidirectional fluidic channel; and an eluant for eluting the at least one material of interest from the multidirectional fluidic channel.

In some embodiments of the kit, the substance is coating at least a portion of the inside of the at least one wall of the multi-directional fluidic channel. In some embodiments of the kit, the substance is configured to adsorb one or more materials of interest. In some embodiments of the kit, the eluant is an elution buffer. In some embodiments of the kit, the eluant is a lysis buffer. In some embodiments of the kit, the substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments of the kit, one or more substances or a plurality of the substances are coated on at least one wall of the multi-directional fluidic channel, wherein each substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent. In some embodiments of the kit, the substance coating the channel wall comprises a fixed, covalently-bonded polypeptide antibiotic. In some embodiments of the kit, the fixed, covalently-bonded polypeptide antibiotic is polymyxin. In some embodiments of the kit, an amount of polymyxin fixed is at least 0.5 mM. In some embodiments of the kit, the amount of polymyxin fixed is about 1.0 to about 50.0 mM. In some embodiments of the kit, the fixed, covalently-bonded polypeptide antibiotic is vancomycin. In some embodiments of the kit, an amount of vancomycin fixed is at least 0.5 mM. In some embodiments of the kit, the amount of vancomycin fixed is about 1.0 to about 50.0 mM. In some embodiments of the kit, the multi-directional fluidic channel is composed of at least one thermoplastic polymer base material having at least one surface-exposed functional group. In some embodiments of the kit, the thermoplastic polymer base material comprises at least one exposed surface selected from the functional group consisting of: a carbonyl group, a carboxyl group, an alcohol group, an amino group, a chloride group, a styrene group, an alpha-halogenated acyl group, a benzyl group, and an isocyanic acid group, and a remaining thermoplastic polymer base material further comprises other polymers or copolymers. In some embodiments of the kit, the remaining thermoplastic polymer base materials comprise: a polyvinyl chloride; a polyvinyl acetate; a polyvinyl benzene; a polytetrafluoroethylene; a polyamide; an acrylamide; a polyurethane; a polyethylene; a polyethylene terephthalate; a polydimethylsiloxane; a polyacrylonitrile; a polycarbonate; an acetal; a polyethylene; a polypropylene; or a polymethyl methacrylate. In some embodiments of the kit, the thermoplastic polymer base material is polycarbonate. In some embodiments of the kit, the thermoplastic polymer base material is polymethyl methacrylate (PMMA). In some embodiments of the kit, the fluidic cartridge is disposable. In some embodiments of the kit, a crosslinking agent to be fixed to the base material is polyethylene glycol or a derivative substance. In some embodiments of the kit, an amount of the crosslinking agent to be fixed is about 1 mM to about 50 mM. In some embodiments of the kit, an amount of the crosslinking agent to be fixed is at least about 1 mM. In some embodiments of the kit, an amount of the crosslinking agent to be fixed is at most about 50 mM.

It should further be appreciated that any of the fluidic cartridges or methods described or contemplated herein may be used in series or parallel.

### EXAMPLES

Following sample collection, qPCR was performed. The Qiagen qPCR Array for microbial DNA testing kit can be used. To complete the Microbial DNA qPCR Array procedure, the collected sample DNA from operation 1538 of the method 1500 was mixed with the ready-to-use Qiagen Microbial qPCR Mastermix and the mixture was aliquoted into each desired well of the Qiagen 96-well kit plate containing pre-dispensed, gene-specific primer and hydrolysis probe sets. qPCR was then performed according to the recommended cycling conditions.

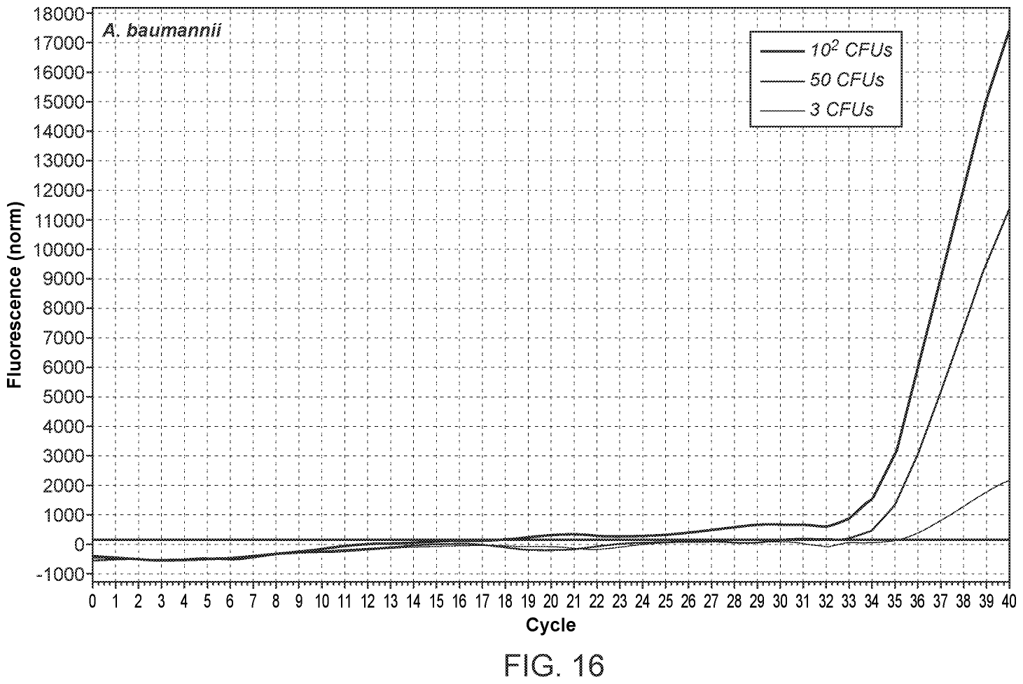

FIG. 16 shows an example of rapid Acinetobacter baumannnii detection based on real-time qPCR. Bacteria real-time qPCR reaction was performed after flowing bacteria in whole blood through device 900 in the amount of 10² CFUs (colony forming units), 50 CFUs, and 3 CFUs—corresponding to high, intermediate, and low bacterial concentrations, respectively. Following operation 1538 of the method 1500, the samples were analyzed using qPCR. The data were analyzed from cycle 2 to 40.

In another example, following DNA sample collection from operation 1538 of the method 1500 and subsequent DNA amplification by PCR, nanopore sequencing may be performed using the Oxford Nanopore MinION. MinION sequencing libraries are prepared from PCR amplicons. Genomic DNA sequencing may be performed according to the manufacturer's recommendation.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for the capture and isolation of materials of interest comprising:
flowing a sample through a fluidic cartridge comprising a multi-directional fluidic channel between an inlet and an outlet;
adsorbing one or more materials of interest on at least one wall of the multi-directional fluidic channel;
washing the multidirectional fluidic cartridge channel with a wash buffer to separate the adsorbed materials of interest from a residual sample matter that is not of interest;
discarding said wash buffer containing the residual sample matter that is not of interest;
flowing an eluant through the fluidic cartridge containing the adsorbed materials of interest on the at least one wall in the multi-directional fluidic channel between the inlet and the outlet, collecting and concentrating the materials of interest in an eluate; and
collecting an amount of the eluate containing the one or more concentrated materials of interest eluted from the sample.

2. The method of claim 1, wherein the sample comprises an additive before flowing through the fluidic cartridge.

3. The method of claim 1 or 2, wherein a substance is coated on the at least one wall of the multi-directional fluidic channel.

4. The method of claim 1 or 2 wherein, the eluant is an elution buffer.

5. The method of claim 1 or 2 wherein, the eluant is a lysis buffer.

6. The method of claim 3, wherein the substance is selected from the group consisting of:
an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent.

7. The method of claim 1 or 2, wherein one or more substances or a plurality of the substances are coated on at least one wall of the multi-directional fluidic channel, wherein each substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent.

8. The method of claim 6 or 7, wherein the substance coating the channel wall comprises a fixed, covalently-bonded polypeptide antibiotic.

9. The method of claim 8, wherein the fixed, covalently-bonded polypeptide antibiotic is polymyxin.

10. The method of claim 9, wherein an amount of polymyxin fixed is at least 0.5 mM.

11. The method of claim 10, wherein the amount of polymyxin fixed is about 1.0 to about 50.0 mM.

12. The method of claim 8, wherein the fixed, covalently-bonded polypeptide antibiotic is vancomycin.

13. The method of claim 12, wherein an amount of vancomycin fixed is at least 0.5 mM.

14. The method of claim 13, wherein the amount of vancomycin fixed is about 1.0 to about 50.0 mM.

15. The method of claim 1, wherein the multi-directional fluidic channel is composed of at least one thermoplastic polymer base material having at least one surface-exposed functional group.

16. The method of claim 15, wherein the thermoplastic polymer base material comprises at least one exposed surface selected from the functional group consisting of:
a carbonyl group, a carboxyl group, an alcohol group, an amino group, a chloride group, a styrene group, an alpha-halogenated acyl group, a benzyl group, and an isocyanic acid group, and
a remaining thermoplastic polymer base material further comprises other polymers or copolymers.

17. The method of claim 16, wherein the remaining thermoplastic polymer base materials comprise:
a polyvinyl chloride, a polyvinyl acetate, a polyvinyl benzene,
a polytetrafluoroethylene, a polyamide, an acrylamide, a polyurethane, a polyethylene, a polyethylene terephthalate, a polydimethylsiloxane, a polyacrylonitrile, a polycarbonate, an acetal plastic, a polyethylene, a polypropylene, or a polymethyl methacrylate.

18. The method of claim 16, wherein the thermoplastic polymer base material is polycarbonate.

19. The method of claim 15, wherein a crosslinking agent to be fixed to the base material is polyethylene glycol or a derivative substance.

20. The method of claim 19, wherein an amount of crosslinking agent to be fixed is about 1 mM to about 50 mM.

21. The method of claim 19, wherein an amount of crosslinking agent to be fixed is at least about 1 mM.

22. The method of claim 19, wherein an amount of crosslinking agent to be fixed is at most about 50 mM.

23. The method of claim 1, wherein the fluidic cartridge is disposable.

24. The method in claim 1, wherein the multi-directional channel has an internal width of about 0.001 to about 100.0 mm.

25. The method of claim 24, wherein the multi-directional channel has an internal width of about 0.01 to about 10.0 mm.

26. The method in claim 1, wherein the multi-directional channel has an internal height of about 0.005 to about 50.0 mm.

27. The method of claim 26, wherein the multi-directional channel has an internal height of about 0.05 to about 5.0 mm.

28. The method in claim 1 wherein the multi-directional channel has a length of 0.01 to 10,000 mm between the inlet and outlet.

29. The method of any one of claims 24-28, wherein the multi-directional channel has a length of about 0.1 to about 1000.0 mm between the inlet and outlet.

30. The method of claim 1, wherein said multi-directional channel is spiral shaped.

31. The method of claim 30, wherein the spiral shaped channel has a radius of curvature ranging between about 0.01 to about 1000.0 mm.

32. The method of claim 31, wherein the spiral shaped channel has a radius of curvature ranging between about 0.1 to about 100.0 mm.

33. The method of claim 30, wherein the spiral shaped multi-directional channel has a distance between channel edges in the spiral of about 0.01 to about 1,000 mm.

34. The method of claim 33, wherein the spiral shaped multi-directional channel has a distance between the channel edges in the spiral of about 0.1 to about 10.0 mm.

35. The method of claim 1, wherein said multi-directional channel is helically shaped and fabricated around a cylindrical chamber.

36. The method of claim 35, wherein the helical shaped multi-directional channel has a radius of curvature ranging between about 1.0 to about 1,000.0 mm.

37. The method of claim 36, wherein the helical shaped multi-directional channel has a radius of curvature ranging between about 5.0 to about 100.0 mm.

38. The method of claim 37, wherein the helical shaped multi-directional channel has a pitch ranging between about 1.0 to about 1,000.0 mm.

39. The method of claim 38, wherein the helical shaped multi-directional channel has a pitch ranging between about 10.0 to about 100.0 mm.

40. The method of claim 1, wherein the multi-directional channel comprises at least one inlet and at least one outlet.

41. The method of claim 1, wherein the fluidic cartridge is a multi-part assembly enclosed by using at least one of:
bolts;
an adhesive;
a binding material;
a resin;
an inner sleeve on a cylinder; and
an outer sleeve on a cylinder;
utilizing at least one of:
a base plate;
a cover glass;
a curing (method);
a thermal expansion (process);
ultrasonic welding;
vibration welding;
high frequency welding; (aka: radio frequency welding, and dielectric welding)
heated tool or plate welding;
solvent bonding;
laser welding;
spin welding;
infrared welding; and
adhesive bonding.

42. The method of claim 1, wherein the fluidic cartridge comprising the multi-directional channel is fabricated using at least one method selected from the group consisting of:
3-D printing;
stereolithography;
photolithography
injection molding;
blow molding;
casting;
ultrasonic welding;
vibration welding;
high frequency welding; (aka: radio frequency welding, and dielectric welding)
heated tool or plate welding;
solvent bonding;
laser welding;
spin welding;
infrared welding;
adhesive bonding;
machining;
turning;
drilling;
boring;
reaming;
electric discharge machining (EDM); and
milling.

43. The method of claim 1 or 40, wherein syringes are attached to the at least one multi-directional channel inlet or at least one outlet with:
fittings;
caps; or
luer lock connectors.

44. The method of claim 1 wherein the eluant is selected from a group comprising:
Tris-HCl;
Ethylenediaminetetraacetic acid (EDTA);
sodium dodecyl sulfate (SDS);
TritonX-100;
a chaotropic buffer;
proteinase K;
sodium heparin;
a heparin compound;
fluoride;
oxalate;
sodium citrate;
sodium polyanethol sulfonate (SPS);
Acid Citrate Dextrose Solution;
distilled H₂O; or
saline.

45. The method of claim 44, wherein the eluant contains 0.001M-100M of Tris-HCL.

46. The method of claim 44, wherein the eluant has a pH between 4-11.

47. The method of claim 44, wherein the eluant contains 0.001M-100M of EDTA, having a pH between 6-9.

48. The method of claim 44, wherein the eluant contains 0.01%-90% of sodium dodecyl sulfate, having a pH between 6-9.

49. The method of claim 44, wherein the eluant contains 0.01%-90% of TritonX-100, having a pH between 6-9.

50. The method of claim 44, wherein the eluant contains 0.001M-100M of a chaotropic buffer, having a pH between 6-9.

51. The method of claim 48, wherein the eluant is Guanidinium thiocyanate.

52. The method of claim 47, wherein the eluant contains 0.001-100 mg/mL of proteinase K, having a pH between 6-9.

53. The method of claim 1, wherein the concentrated eluted material of interest is washed and purified by placing said eluant in a spin column and placed in a centrifuge for centrifugation.

54. The method of claim 53, wherein a purified target material(s) is entrapped in a spin column matrix, while other unwanted materials pass through the spin column matrix into the collection vial and discarded. The targeted sample material entrapped

55. The method of claim 53, wherein the entrapped purified target material(s) in the spin column matrix is released from the matrix by washing said matrix with a buffer solution and the released purified target material is collected into a clean collection vial.

56. The method of claim 55, wherein the washed eluted material of interest is analyzed using at least one of:
a polymerase chain reaction (PCR);
a matrix-assisted laser desorption/ionization-time of flight, (MALDI-TOF);
a nuclear magnetic resonance (NMR) spectroscopy;
culturing;
a fluorescence in situ hybridization (FISH);
optically active microbeads; and
optically active nanoparticles.

57. The method of claim 56, wherein the eluted material of interest is analyzed for the presence or an amount of the one or more specific materials of interest.

58. The method of claim 2, wherein the additive comprises one or more of:
an EDTA;
a K₂EDTA;
a heparin compound;
a fluoride;
an oxalate;
a sodium citrate;
a sodium polyanethol sulfonate (SPS);
an Acid Citrate Dextrose Solution;
a distilled H2O; or
a saline.

59. The method of claim 55, wherein the washing is performed with a buffer solution comprising one or more of:
a saline;
an ethyl alcohol;
a sterile H₂O; or
a combination thereof.

60. A kit for the capture and adsorption of materials of interest comprising:
a fluidic cartridge with an inlet and an outlet;
a multidirectional fluidic channel between the inlet and the outlet;
said multidirectional fluidic channel comprising:
at least one wall;
a substance coating at least one wall of the multidirectional fluidic channel; and
an eluant for eluting at least one material of interest from the multidirectional fluidic channel.

61. The kit of claim 60, wherein the substance is coating at least a portion of the at least one wall of the multi-directional fluidic channel.

62. The kit of claim 61, wherein the substance is configured to adsorb one or more materials of interest.

63. The kit of claim 60, wherein the eluant is an elution buffer.

64. The kit of claim 60, wherein the eluant is a lysis buffer.

65. The kit of claim 62, wherein the substance is selected from the group consisting of:
an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent.

66. The kit of claim 62, wherein one or more substances or a plurality of the substances are coated on at least one wall of the multi-directional fluidic channel, wherein each substance is selected from the group consisting of: an antibody, a crosslinking agent, a peptide, a protein, an antibiotic, a polymer, an amine, a polyether, an amino acid, an aptamer, a tumor necrosis factor, an adhesion receptor, an E-selectin, a cytokine, a chemotherapy agent, a quorum sensing protein, a quorum sensing receptor, a polysaccharide, and a biological agent.

67. The kit of claim 65 or 66, wherein the substance coating the channel wall comprises a fixed, covalently-bonded polypeptide antibiotic.

68. The kit of claim 67, wherein the fixed, covalently-bonded polypeptide antibiotic is polymyxin.

69. The kit of claim 68, wherein an amount of polymyxin fixed is at least 0.5 mM.

70. The kit of claim 69, wherein the amount of polymyxin fixed is about 1.0 to about 50.0 mM.

71. The kit of claim 67, wherein the fixed, covalently-bonded polypeptide antibiotic is vancomycin.

72. The kit of claim 71, wherein an amount of vancomycin fixed is at least 0.5 mM.

73. The kit of claim 72, wherein the amount of vancomycin fixed is about 1.0 to about 50.0 mM.

74. The kit of claim 60, wherein the multi-directional fluidic channel is composed of at least one thermoplastic polymer base material having at least one surface-exposed functional group.

75. The kit of claim 74, wherein the thermoplastic polymer base material comprises at least one exposed surface selected from the functional group consisting of:
a carbonyl group, a carboxyl group, an alcohol group, an amino group, a chloride group, a styrene group, an alpha-halogenated acyl group, a benzyl group, and an isocyanic acid group, and
a remaining thermoplastic polymer base material further comprises other polymers or copolymers.

76. The kit of claim 75, wherein the remaining thermoplastic polymer base materials comprise:
a polyvinyl chloride;
a polyvinyl acetate;
a polyvinyl benzene;
a polytetrafluoroethylene;
a polyamide;
an acrylamide;
a polyurethane;
a polyethylene;
a polyethylene terephthalate;
a polydimethylsiloxane;
a polyacrylonitrile;
a polycarbonate;
an acetal plastic;
a polyethylene;
a polypropylene; or
a polymethyl methacrylate.

77. The kit of claim 75, wherein the thermoplastic polymer base material is polycarbonate.

78. The kit of claim 75, wherein the thermoplastic polymer base material is polymethyl methacrylate (PMMA).

79. The kit of claim 60, wherein the fluidic cartridge is disposable.

80. The kit of claim 74, wherein a crosslinking agent to be fixed to the base material is polyethylene glycol or a derivative substance.

81. The kit of claim 80, wherein an amount of the crosslinking agent to be fixed is about 1 mM to about 50 mM.

82. The kit of claim 80, wherein an amount of the crosslinking agent to be fixed is at least about 1 mM.

83. The kit of claim 80, wherein an amount of the crosslinking agent to be fixed is at most about 50 mM.
